(19) United States
(12) Patent Application Publication
(10) Pub. No.: US 2026/0027228 A1
(43) Pub. Date: Jan. 29, 2026

(54) DISPERSING SPECIFIC BIOMOLECULAR CONDENSATES THROUGH MOLECULAR CHAPERONES

(71) Applicant: University of Virginia Patent Foundation, Charlottesville, VA (US)

(72) Inventor: Hao Jiang, Charlottesville, VA (US)

(21) Appl. No.: 19/260,688

(22) Filed: Jul. 7, 2025

Related U.S. Application Data
(60) Provisional application No. 63/675,770, filed on Jul. 26, 2024.

(51) Int. Cl. A61K 47/68; C07K 14/435

(52) U.S. Cl. CPC A61K 47/6851; C07K 14/435; C07K 2319/30

(57) ABSTRACT

In one aspect, the disclosure relates to compounds methods for treating or preventing diseases associated with aberrant condensation of a biomolecule in a subject, the method including at least the step of contacting one or more cells in the subject with a fusion protein that includes a J domain protein and a targeting molecule, wherein the targeting molecule binds the biomolecule. In one aspect, the biomolecule can be a target protein that may be mutated and/or include one or more intrinsically disordered regions. In another aspect, the targeting molecule can be a nanobody, but other targeting molecules are also contemplated. In still another aspect, the disclosed method is useful for treating and/or preventing cancers such as blood cancers and non-small cell lung cancer. Also disclosed are methods for disrupting condensates in cell culture.

### CROSS-REFERENCE TO RELATED APPLICATIONS

This application claims the benefit of U.S. Provisional Application Ser. No. 63/675,770, filed Jul. 26, 2024, which is incorporated herein by reference in its entirety.

### CROSS-REFERENCE TO SEQUENCE LISTING

This application contains a sequence listing filed in ST.26 format entitled “222117-1570_Sequence_Listing.xml” created on Jun. 10, 2025, and having a file size of 6,998 bytes. The content of the sequence listing is incorporated herein in its entirety.

### BACKGROUND

Biomolecular condensates are membraneless micron-scale assemblies in cells that function to concentrate biomolecules. They are assembled by multivalent and weak intermolecular interactions of proteins often featuring intrinsically disordered regions (IDRs) or modular domains, as well as nucleic acids. They can adopt a broad and continuous spectrum of material properties, from highly dynamic liquid droplets to less dynamic gels and solid aggregates. As fundamental organizers of cellular biochemistry, biomolecular condensates are involved in virtually all cellular processes, including but beyond signaling, chromatin organization, and gene expression. Alterations in condensate formation, dissolution, and properties are associated with a wide range of disease, including cancer, neurodegenerative disease, metabolic disorder, infection, and inflammation.

Despite the rapid development in this field, some fundamental questions remain outstanding: What functional roles do biomolecular condensates play in a physiological or pathological process? What are the molecular mechanisms by which condensates regulate a cellular process? Can condensates of a specific endogenous protein be dispersed to control the biological or disease process the condensates are involved in? To address these questions, the endogenous condensates in cells should be specifically perturbed or disrupted. While IDRs are difficult to target, several studies have identified molecules to perturb, disperse, or modify condensates, or their properties. These molecules may affect condensates through unknown mechanisms or based on specific physicochemical properties, which are often shared by other proteins. Therefore, the off-target effects of these non-specific approaches can be an issue. To date, targeted dispersal/disassembly of specific, naturally occurring, endogenous protein condensates in cells has not been reported.

ASXL1 mutations are very frequent (15-50%) in all forms of myeloid malignancies and always associated with adverse prognosis, relapse, and therapy resistance. ASXL1 is an epigenetic regulator that facilitates histone H2A deubiquitylation. ASXL1 mutations are mostly C-terminal truncations and gain-of-function to promote malignancies, but the underlying mechanisms are poorly understood. It was found that the ASXL1 N-terminal region has an intrinsic phase separation property (forming liquid-like condensates) that is normally suppressed by the cancer-deleted sequences in the wild type ASXL1 in cells. The frequent ASXL1 truncating mutants escape from this regulatory mechanism and unleash the condensation property to promote leukemogenesis via epigenetic dysregulation. It would thus be desirable to target the condensates of ASXL1 mutants to inhibit myeloid cancer.

Despite advances in blood cancer research, the mechanism by which ASXL1 mutations cause blood cancers is poorly understood, and there is thus currently no strategy to target ASXL1 mutants. It would be desirable to target dispersal/disassembly of specific, naturally occurring, endogenous protein condensates in cancer cells having ASXL1 mutations in order to treat myeloid and other malignancies. It would further be desirable to develop a generalizable method for targeting endogenous condensates of other wild-type and mutant proteins active in additional condensate-driven pathological processes. These needs and other needs are satisfied by the present disclosure.

### SUMMARY

In accordance with the purpose(s) of the present disclosure, as embodied and broadly described herein, the disclosure, in one aspect, relates to compounds methods for treating or preventing diseases associated with aberrant condensation of a biomolecule in a subject, the method including at least the step of contacting one or more cells in the subject with a fusion protein that includes a J domain protein and a targeting molecule, wherein the targeting molecule binds the biomolecule. In one aspect, the biomolecule can be a target protein that may be mutated and/or include one or more intrinsically disordered regions. In another aspect, the targeting molecule can be a nanobody, but other targeting molecules are also contemplated. In still another aspect, the disclosed method is useful for treating and/or preventing cancers such as blood cancers and non-small cell lung cancer. Also disclosed are methods for disrupting condensates in cell culture.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1B show that J-domain fusion disperses different protein condensates. (FIG. 1A) Representative fluorescence microscopy images of 293T cells transfected with indicated EGFP-tagged target proteins, or these proteins fused with J or J^{H33Q}. ND, not done. (FIG. 1B) A cartoon model for this figure.

FIGS. 2A-2E show J-domain fusion disrupts biological function of the condensates. (FIG. 2A) Reporter splicing efficiency, measured by double reporter splicing assays in 293T cells co-transfected with indicated siRNA and rescue constructs (two doses of J- or J^{H33Q}-AKAP95). Top, immunoblotting for expression of the construct and endogenous AKAP95. AKP=AKAP95. (FIG. 2B) Immunoblotting and growth for MDA-MB-231 cells co-transfected with indicated siRNA (left) or shRNA (right) and rescue constructs. J-AKP sample was underloaded in the left blot. The siRNA effects wore out after day 2 but the shRNA effects lasted longer. (FIG. 2C) Activity of UTX, or UTX fused with J or J^{H33Q} in H3K27 demethylation in transfected cells. (FIGS. 2D-2E) ASXL1^{G646Wfs*12} activity in H2A deubiquitination. 293T cells were transfected with ASXL1^{G646Wfs*12} that was not fused, or fused with J or J^{H33Q}. (FIG. 2D) Representative immunofluorescence images of cells stained for ASXL1 and H2AK119 ubiquitination. White circled cells expressed the ASXL1 construct, and reduction of H2Aub signals in these cells compared other cells indicate active deubiquitination. Also note the ASXL1 puncta in No J fusion and fusion with J^{H33Q} cells, but not in the fusion with J cells. (FIG. 2E) Percentage of transfected cells that showed H2Aub reduction. In bars, number of cells showing H2Aub reduction/number of transfected cells counted, in three biological repeats. P values by Student's t-test.

FIGS. 3A-3B show J-nb disperses target condensates. (FIG. 3A) Representative immunofluorescence images of 293T cells co-transfected with indicated EGFP-tagged target proteins and fusion of J^{H33Q}- or J- with GFPnb-mCherry. (FIG. 3B) Representative immunofluorescence images of 293T cells co-transfected with ALFA-EGFP-tagged MLL-AF9 and mCherry-tagged ALFAnb (top two rows) or ALFAnbmt (ALFAnb with multiple mutations to disrupt ALFA binding) (bottom two rows), or nb fusion with J or J^{H33Q}.

FIGS. 4A-4G show J-nb disperses MLL-AF9 condensates and disrupts its tumorigenicity and transcriptional activity. (FIG. 4A) The experimental scheme. (FIGS. 4B-4C and 4E-4G) BM was co-transduced with MLL-AF9-ALFA-GFP and (no J, J, or J^{H33Q})-ALFAnb-mCherry, as shown in (FIG. 4A). (FIG. 4B) Representative fluorescence microscopy images of the GFP⁺mCherry⁺cells expressing indicated ALFAnb variant constructs. (FIG. 4C) Same number of GFP⁺mCherry⁺cells were serially replated for colony formation. Representative colony images of 1^{st} and 2^{nd} plating and the entire 35 mm-dish view of the 2^{nd} plating. Bar graph, number of the colonies. 2 repeats for 1^{st} and 3 repeats for 2^{nd} round. (FIG. 4D) MLL-AF9-ALFA-GFP-transduced BM was transplanted into primary recipients to develop leukemia. BM from these leukemic mice was transduced with (J or J^{H33Q})-ALFAnb-mCherry, and the sorted GFP⁺mCherry⁺ cells were further transplanted to secondary recipients, as shown in (FIG. 4A). Representative images of Giemsa staining of the peripheral blood cells derived from these secondary recipients. Insets show zoomed-in images of the cells for clearer morphological differences. Right, plot of the percent of leukemic blasts. N=7 mice each. (FIGS. 4E-4G) RNA-seq analysis of GFP⁺mCherry⁺ BM co-transduced with MLL-AF9-ALFA-GFP and (no J, J, or J^{H33Q})-ALFAnb-mCherry, as shown in (FIG. 4A). (FIG. 4E) Heatmap showing relative expression levels, in all three BMs co-transduced with MLL-AF9-ALFA-GFP and (no J, J, or J^{H33Q})-ALFAnb-mCherry, of 614 genes that were differentially expressed (logFC>5 or <−5, and FDR<0.00001) between co-transduction with J-ALFAnb versus J^{H33Q}-ALFAnb. (FIG. 4F) GSEA analysis of genes downregulated by J-ALFAnb versus J^{H33Q}-ALFAnb as compared with the MLL-AF9 regulated genes or MLL-AF9 highly occupied genes. MLL-AF9 regulated genes are defined as genes with 25% or greater reduction of expression after 3 h of treatment that degrades MLL-AF9 and differentially expressed genes (up or down, FC>2, p<0.05) after 24 h of treatment that degrades MLL-AF9. MLL-AF9 highly occupied genes are defined as genes with a disproportionally high binding of MLL-AF9 around the promoter in HA-MLL-AF9 ChIP-seq assays. The heatmap shows genes comprising the leading edge of the GSEA plot as compared with the MLL-AF9 regulated genes. (FIG. 4G) Expression levels (normalized read counts in RNA-seq) of indicated MLL-AF9 target genes in BMs co-transduced with MLL-AF9-ALFA-GFP and (no J, J, or J^{H33Q})-ALFAnb-mCherry. P<=0.01 for all of these genes except Hoxa6 (P<0.05) between J-ALFAnb and J^{H33Q}-ALFAnb. P values by Student's t-test. *P≤0.05, **P≤0.01, ***P≤0.001, ****P≤0.0001. ns, P>0.05.

FIGS. 5A-5B show J-nb recruits Hsp70s to disperse condensates in the in vitro reconstituted system. ASXL1 (1-590)-mEGFP was co-incubated with either mCherry or the dispersal machinery that contained mCherry-HSPA8, mCherry-BAG2, and (J/J^{H33Q}/no J)-GFPnb proteins, under+ATP (FIG. 5A) or −ATP (FIG. 5B) conditions, and imaged by fluorescence microscopy. DIC, differential interference contrast microscopy. Scale bar, 5 μm.

FIGS. 6A-6G show dispersing and inactivating endogenous, unmodified condensates by a target-specific nanobody. (FIG. 6A) Representative fluorescence microscopy images of 293T cells transfected with GFP-tagged c-FOS, or c-FOS fused with J or J^{H33Q}. (FIG. 6B) Representative fluorescence microscopy images of 293T cells co-transfected with GFP-tagged c-FOS and mCherry-tagged J- or J^{H33Q}-FOSnb. (FIG. 6C) K562 cells were transduced with J- or J^{H33Q}-fused FOSnb-mCherry, and treated with PMA to induce endogenous c-FOS. Representative immunofluorescence images of mCherry and induced c-FOS (by α-c-FOS staining). Note the c-FOS signals were diffused in the arrow-pointed cells that expressed the J-FOSnb, but showed discrete puncta in the arrow-pointed cells that expressed J^{H33Q}-FOSnb. c-FOS puncta were prevalent in the rest of the cells (no arrows) in both image that did not express the J- or J^{H33Q}-fused FOSnb. (FIGS. 6D-6E) Giemsa staining of K562 cells stably expressing indicated constructs and treated with PMA (or not). Cells with megakaryocyte-like features (extensive vacuolation) were quantified (right). Note in FIG. 6E, lack of effects by J-fusion with non-targeting nb (ALFAnb, as K562 cells have no ALFA-tagged proteins). Each sample has indicated color consistent between the staining image and the plot. N=6 repeats. (FIG. 6F) Giemsa staining of K562 cells treated with purified TAT-J-FOSnb or TAT-J^{H33Q}-FOSnb proteins. N=3 repeats for no treatment and 4 repeats for each of the other samples. (FIG. 6G) A cartoon model for this figure. P values by Student's t-test.

FIGS. 7A-7H show dispersing and inactivating unmodified, endogenous condensates by a target-binding protein or domain. (FIGS. 7A-7C) Representative fluorescence microscopy images of 293T cells co-transfected with GFP-tagged MLL-AF9 and mCherry-tagged Menin (Men) (FIG. 7A), Menin^{Y323K} (FIG. 7B), or LEDGF_{IBD} (IBD) (FIG. 7C), or their J- orJ^{H33Q} fusion. Scale bar, 5 μm. (FIGS. 7D-7F) Growth curves of indicated leukemia cell lines (driving mutation in parenthesis) that stably expressed Menin (FIG. 7D), or LEDGF_{IBD} (FIG. 7E), or Menin^{Y323K} (FIG. 7F), or their J- or J^{H33Q} fusion, as well as controls, as indicated. N=3 repeats. (FIG. 7G) Relative expression of indicated MLL-AF9 target genes in MOLM-13 cells that stably expressed indicated constructs, as determined by RT-qPCR assays and normalized to GAPDH. N=2 repeats. (FIG. 7H) A cartoon model for this figure. P values by Student's t-test. *P≤0.05, **P≤0.01, ***P≤0.001, ****P≤0.0001.

FIGS. 8A-8C show J-domain fusion does not affect target binding with their binding partners. (FIG. 8A) Fluorescence intensity of indicated proteins unfused or fused with J or J^{H33Q} (all GFP-tagged). 293T cells transfected with indicated constructs and the GFP fluorescence signals were quantified by ImageJ by measuring the average signal intensity in each cell. Cell images were randomly selected. Number of cells analyzed is labeled at the bottom. (FIGS. 8B-8C) Immunoprecipitation by FLAG for the FLAG-HA-tagged (FH-) J- or J^{H33Q}-fused UTX (FIG. 8B) and AKAP95 (FIG. 8C) from transfected 293T cells, followed by immunoblotting for Hsp70 and RbBP5 (of MLL3/4 complex) for UTX or DDX5/p68 for AKAP95.

FIGS. 9A-9E show J-nb disperses MLL-AF9 condensates and disrupts its tumorigenicity. (FIG. 9A) Disorder plot and domain structure of MLL-AF9. MLL portion of the fusion (MLL_{f}) is in blue and AF9 is in purple. H-T-H, helix-turn-helix. (FIG. 9B) Representative fluorescence microscopy images of 293T cells transfected with GFP-tagged MLL-AF9 or MLL_{f}. (FIG. 9C) Representative fluorescence microscopy images of 293T cells transfected with indicated EGFP-tagged MLL-AF9, or its fusion with J or J^{H33Q}. (FIG. 9D) Immunoprecipitation by FLAG for the FLAG-HA-tagged (FH-) J- or J^{H33Q}-fused MLL-AF9-GFP from transfected 293T cells, followed by immunoblotting for Hsp70 and Menin. Two doses of J^{H33Q}-fusion plasmid were used to ensure similar expression as J-fusion. (FIG. 9E) Mouse BM was co-transduced with MLL-AF9-ALFA-GFP and mCherry-tagged ALFAnb or ALFAnbmt, or their fusion with J or J^{H33Q}. Same number of sorted GFP⁺mCherry⁺ cells were plated for colony formation. Bar graph, normalized number of the colonies. 2 repeats. Bottom, representative colony images.

FIG. 10 shows J-nb disrupts MLL-AF9's tumorigenicity. BM from MLL-AF9-ALFA-GFP leukemic mice (derived from transplant of MLL-AF9-ALFA-GFP-transduced BM) was transduced with (J or J^{H33Q})-ALFAnb-mCherry, and the sorted GFP⁺mCherry⁺ cells were further transplanted to secondary recipients, as shown in FIG. 4A. Counts of various types of blood cells as indicated in the peripheral blood of the secondary recipient mice at 3 months after transplant. Each dot represents a mouse. WBC, white blood cells. NE, neutrophils. LY, lymphocytes. MO, monocytes. EO, eosinophils. BA, basophils. RBC, red blood cells. Data are median (horizontal line), 25-75th percentiles (box) and 1.5 times the interquartile range recorded (whiskers), and the black dashed line indicates average. P values by Student's t-test.

FIGS. 11A-11D show J-nb disrupts MLL-AF9's activity in target gene transcription. GSEA analysis of genes downregulated by J-ALFAnb versus J^{H33Q}-ALFAnb (FIGS. 11A-11B) or versus ALFAnb (FIGS. 11C-11D), as compared with genes overexpressed in MLL-rearranged pediatric acute myeloid leukemias from two different resources (FIGS. 11A and 11C) or (FIGS. 11B and 11D). The heatmaps show genes comprising the leading edge of the GSEA plots.

FIGS. 12A-12H show the in vitro reconstituted system for J-nb to recruit Hsp70s and disperse condensates. (FIG. 12A) Coomassie blue staining images of purified mEGFP and mEGFP-tagged ASXL1 (1-590) proteins. In FIGS. 12A, 12C, 12E, and 12F, two increasing amounts of each protein were loaded, and the red arrows indicate the desired proteins. (FIG. 12B) Representative fluorescence (top) and DIC (bottom) microscopy images of 5 μM purified ASXL1 (1-590) in the condensation assay. (FIG. 12C) Coomassie blue staining images of purified mCherry and mCherry-tagged HSPA8 proteins. Note that, in this panel and panel E, as observed and explained previously, a band of unknown identity, indicated by the asterisk, always appeared at ˜15 kD smaller than the purified mCherry or mCherry-tagged protein of interest, regardless of the identity of the protein of interest. It is thus speculated that it is either a degradation product of mCherry or an irrelevant protein tightly associated with mCherry. (FIG. 12D) Representative fluorescence (top) and DIC (bottom) microscopy images of 2 μM purified HSPA8 in the condensation assay. (FIGS. 12E-12F) Coomassie blue staining images of purified mCherry-tagged BAG2 (FIG. 12E), and (untagged) GFPnb, or GFPnb fused with J or J^{H33Q} proteins. Note the GFPnb is composed of two GFPnb linked in tandem for enhanced binding affinity, thus giving a higher molecular weight than a single nb. It is also possible that the linker regions between the two GFPnb units was more susceptible to protease digestion, generating the degradation product indicated by the asterisk. (FIGS. 12G-12H) As a control, mEGFP was co-incubated with the dispersal machinery that contained mCherry-HSPA8, mCherry-BAG2, and (J/J^{H33Q}/no J)-GFPnb proteins, under+ATP (FIG. 12G) or −ATP (FIG. 12H) conditions, and imaged by fluorescence microscopy. DIC, differential interference contrast microscopy. Scale bar, 5 μm.

FIGS. 13A-13F show dispersing and inactivating unmodified, endogenous c-FOS condensates. (FIG. 13A) Representative fluorescence microscopy images of 293T cells transfected with GFP-tagged c-FOS. (FIG. 13B) Immunoblotting for c-FOS in K562 cells untreated or treated with 100 nM PMA. (FIG. 13C) Representative immunofluorescence (left two panels) and confocal microscopy images (right two panels) for c-FOS in K562 cells untreated or treated with 100 nM PMA. (FIG. 13D) Immunoprecipitation by FLAG for the FLAG-HA-tagged (FH-) J- or J^{H33Q}-fused c-FOS-GFP from transfected 293T cells, followed by immunoblotting for GFP and c-JUN. Hsp70 signals are seen on the Ponceau S staining. (FIG. 13E) Coomassie blue staining of the purified TAT-mCherry-tagged J-FOSnb or J^{H33Q}-FOSnb proteins. (FIG. 13F) Representative immunofluorescence images of K562 cells untreated or treated with purified TAT-mCherry-tagged J-FOSnb or J^{H33Q}-FOSnb proteins.

FIG. 14 shows dispersing and inactivating unmodified, endogenous condensates by a target-binding protein or domain. Immunoblotting of total lysates from MOLM-13 cells stably expressing mCherry-tagged Menin or LEDG_{IBD}, or their J- or J^{H33Q} fusion.

FIGS. 15A-15F show condensation of ASXL1 truncations promotes H2AK119 deubiquitination. (FIG. 15A) 293T cells expressing EGFP-ASXL1^{G646Wis*12}, its 25RA mutant, or mCherry-BAP1, individually (top) or together as indicated (bottom). (FIG. 15B) Immunostaining for ASXL1 and H2AK119ub in 293T cells transfected with 1-590 variants. Circled: transfected cells. (FIGS. 16C and 16F) Fraction of transfected cells (all 1-590 variants) that showed H2Aub reduction. 3 repeats, 187-280 transfected cells each. (FIGS. 15D-15E) Diagram and images of Δ(391-426), Δ+elF_{IDR}, of 1-590 in 293T cells.

FIGS. 16A-16O show condensation of ASXL1 truncation promotes myeloid malignancies. FIGS. 16A-16E, BM clonogenesis associated assays; FIGS. 16F-16I, engineered K562 assays; FIGS. 16J-16O, leukemogenesis assays. (FIGS. 16A, 16F, and 16J) Assay schematics. (FIGS. 16B-16D) Images and number of colonies (1st plating in FIG. 16C, all three platings in FIG. 16D) for indicated (1-590) variants. 3 repeats. (FIG. 16E) Heatmap for 299 genes upregulated by (1-590) but less efficiently by 25RA in both repeats. (FIG. 16C) Growth for the two +/−clones compared to the parental and sister clones. (FIG. 16H) Growth of clone 20 transduced with indicated (1-590) variants. (FIG. 16I) Western for FIG. 16H. (FIG. 16K) Kaplan-Meier curves for survival of the recipients. P by Logrank test. (FIG. 16L) White blood cell count in peripheral blood of the recipient mice at 9 mon. after transplant. n=4. (FIG. 16M) Giemsa staining of BM cells and quantification of immature blast cells. n=3. (FIG. 16N) Liver and spleen images and spleen weights. n=4 for 1-590, n=1 for control or 10RA at this stage. (FIG. 16O) Flow cytometry analysis of BM.

FIGS. 17A-17I show negative charge in the truncated region regulates ASXL1 condensation and leukemogenic activities. (FIG. 17A) Charge pattern (green box) of ASXL1 . (FIG. 17B) In condensation of 10 μM 646-1067 at indicated pH and net charges. (FIG. 17C) Top, In vitro condensation of 646-1067 WT or indicated mutations at pH 7.4. Bottom: U2OS cells transfected with GFP-tagged 1-1067 and full length (1-1541) that contains WT or indicated mutations in 646-1067. (FIG. 17D) Co-incubation of mEGFP-(1-590) and mCherry-(646-1067) or (646-1067) with 36EQ, pH 7.4. Note the mutual exclusivity of the two colors. (FIGS. 17E-17G) Models for molecular conformations of 1-590, 1-1541, and 1-1541 with mutations in 646-1067. (FIG. 17H) Numbers and images of the colonies from BM transduced with indicated ASXL1 (1-1067) that contains WT or indicated mutations in 646-1067. From 3 repeats. (FIG. 17I) Significant overlap between genes (top) induced more efficiently by 1-1067 with 36EQ in 646-1067 than by 1-1067, and the genes (bottom) induced less efficiently by (1-590) 10RA than 1-590.

FIGS. 18A-18F show dysregulation of condensation is a central mechanism for ASXL1 mutation-associated malignancies. (FIG. 18A) Eight ASXL1 truncating mutations (blue arrows) in myeloid leukemia patients. (FIG. 18B) Top, 293 T cells expressing indicated patient mutants from FIG. 18A. Bottom, in vitro condensation of 10 μM purified ASXL1 proteins all starting from 404 and corresponding to the patient mutants. (FIG. 18C) Number of foci per nucleus (n=11-22 cells each). (FIG. 18D) In vitro partition coefficient for FIG. 18B. (FIG. 18E) H2A deubiquitination by the indicated mutants or WT. 3 repeats. (FIG. 18F) Number of clones from BM expressing indicated mutants or WT. 3 repeats of 2 serial platings.

FIGS. 19A-19C show that receptor tyrosine kinase (RTK) fusions, including EML4-ALK, are important drivers of non-small cell lung cancer (NSCLC). Although tyrosine kinase inhibitors (TKIs) are used as a first-line treatment for NSCLC, resistant mutations often arise. EML4-ALK forms cytoplasmic condensates to drive cancer growth through constitutively active signaling. The strategy of fusing a J-domain with EML4-ALK dissolves the EML4-ALK condensates and disrupts its signaling. FIG. 19A: The most prevalent isoforms of the EML4-ALK fusions in NSCLC. FIG. 19B: testing of J-domain fusions to inhibit EML4-ALK signaling pathways mediated by condensates formed by various isoforms of the EML-ALK protein. FIG. 19C: disrupting EML4-ALK condensates by J-domain fusion in Beas2B cells, an immortalized cell line derived from normal human bronchial epithelium.

FIGS. 20A-20C show targeting unmodified EMLF-ALK condensates disrupts signaling. FIG. 20A: schematic model of condensate disruption. FIG. 20B: disruption of condensates in Beas2B cells. FIG. 20C: The disclosed strategy successfully targets EML4-ALK condensates.

FIGS. 21A-21B show that dissolving endogenous, unmodified EML4-ALK condensates inhibits patient-derived cancer cell growth. FIG. 21A: fluorescence microscopy. FIG. 21B: relative cell number for different J-domain wt and mutant fusion proteins. Cells are NCI-H3122 cells from an EML4-ALK driven NSCLC patient. The R86K mutation disrupts SH2 binding with phosphorylated tyrosine residues.

FIGS. 22A-22B show dissolving condensates of TKI-resistant mutants of EML4-ALK. FIG. 22A: Beas2B cells with 11171N mutation. FIG. 22B: Beas2B cells with G1202R mutation.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The scarcity of loss-of-function tools that can disperse specific endogenous biomolecular condensates is a major barrier to better understanding of condensate functions and treating condensate-driven diseases. By recruiting molecular chaperones through fusing the J-domain with a target-binding nanobody or protein domain, herein is disclosed a strategy that can disperse specific condensates mediated in live cells, including unmodified, naturally occurring, endogenous condensates, and disrupt their functions in driving biological and pathological processes, including cancer and other diseases. In one aspect, in some experiments described herein, it has been shown that J-domain fusion with a MLL-binding nanobody or protein abolished leukemogenesis in mouse and growth of human leukemia cell driven by MLL rearrangements, through dispersing the nuclear condensates and inactivating their target gene expression. In another aspect, J-domain fused to an anti-FOS nanobody dispersed the nuclear condensates of the endogenous, unmodified c-FOS, and inhibited the c-FOS-mediated cellular differentiation process. In any of these aspects, the disclosed method is broadly applicable for studying the functions of biomolecular condensates and controlling condensate-driven pathological processes.

In one aspect, the roles of molecular chaperones in protein quality control and aggregation disassembly are well-known. In another aspect, Hsp70s are central regulators of proteostasis in a broad range of cellular contexts. Further in this aspect, Hsp70s cooperate with co-chaperones, including the J-domain proteins (JDPs/Hsp40) and nucleotide exchange factors (NEFs), as well as other partner chaperones, to constitute a dynamic and versatile Hsp70 chaperone network. In one aspect, the J-domain is a highly conserved ˜70 residue domain required for binding to Hsp70 and stimulating the ATPase activity. In another aspect, JDPs target substrates to Hsp70s and stimulate ATPase to trap substrates, while NEFs then promote ADP release and allow Hsp70s to release the substrates. In an aspect, the term “aggregate” is often used for non-functional and irreversible protein clusters in pathological situations, while “condensate” refers to dynamic, functional, and reversible assemblies. In one aspect these two states are intimately linked, as they are both high-order assemblies with indeterminate stoichiometry. Thus, it is not surprising that chaperones are emerging as critical regulators of formation, dispersal, and core properties of liquid-like biomolecular condensates. In an aspect, Hsp70s (and small HSPs, JDPs) regulate condensation, dynamics, and dispersal of TDP-43 and FUS, disassemble α-synuclein amyloid or Tau fibrils, and also disperse stress-induced biomolecular condensates in yeast.

In an aspect, molecular chaperones regulate biomolecular condensates in cells through the J-domain proteins that bring substrates and activate Hsp70s to disassemble the target protein aggregation and condensation. Herein it is shown that fusion of J-domain with a number of different condensate-forming proteins, including the most frequent ASXL1 leukemia mutant (G646Wfs*12), dissolved the condensates in cells and disrupted their biological functions, suggesting that this strategy may be developed to inactivate cancer-driving condensates.

In one exemplary aspect, fibrolamellar carcinoma is caused by fusion of DNAJB1 J-domain and the catalytic subunit of protein kinase A (J-PKA_{cat}). Hsp70 is recruited to J-PKA_{cat} in the tumors, but the H33Q mutation in the HPD motif in J-domain (J^{H33Q}) disrupts Hsp70 binding. Hsp70 recruitment leads to aberrant signaling for cancer growth. In another aspect, RIα, the type I regulatory subunit of PKA, forms phase-separated condensates critical for cAMP compartmentation. By recruiting Hsp70, J-PKA_{cat} abrogates RIα condensation and causes aberrant cAMP signaling to drive cancer. These results suggest J-domain-mediated Hsp70 recruitment can disperse or disassemble phase-separated liquid-like condensates of endogenous and physiologically functional proteins. In a further aspect, it was reasoned that the Hsp70 activities may be recruited to condensates in the close proximity of a J-domain-containing protein and disperse the condensates. In any of these aspects, in the present disclosure, this mechanism is harnessed to target endogenous proteins to disperse the condensates and disrupt its function.

### Method for Treating or Preventing a Disease Associated with Aberrant Condensation of a Biomolecule

In one aspect, disclosed herein is a method for treating or preventing a disease associated with aberrant condensation of a biomolecule in a subject, the method including at least the step of contacting one or more cells in the subject with a fusion protein. In a further aspect, the fusion protein includes a J domain protein and a targeting molecule. In a still further aspect, the targeting molecule binds the biomolecule. In another aspect, when the targeting molecule binds the biomolecule, the J domain protein recruits a chaperone protein to disassemble the condensate.

In one aspect, the biomolecule can be a target protein, although other biomolecules in addition to peptides and proteins are also contemplated as biomolecules and should be considered disclosed. In some aspects, the target protein can be a truncated mutant, although other types of mutations to wild type sequence and structure are also contemplated and should be considered disclosed including insertions, deletions, repeats, conservative and non-conservative amino acid substitutions, and the like. In some aspects, the target protein can include one or more intrinsically disordered regions.

Exemplary target proteins can include, but are not limited to, MLL-AF9, another MLL-fusion condensate, ASXL1 , EML4-ALK, or any combination thereof. Exemplary chaperone proteins can include, but are not limited to, HSP70. In some aspects, the targeting molecule can be a nanobody or a target protein binding domain.

In an aspect, the subject can be a mammal. such as, for example, a human, non-human primate, dog, cat, horse, cattle, sheep, goat, swine, rabbit, guinea pig, hamster, mouse, or rat. In another aspect, the disease can be a cancer such as, for example, a blood cancer or non-small cell lung cancer (NSCLC). In one aspect, the blood cancer can be leukemia, such as, for example, MLL-rearranged leukemia, and the target protein can be a truncated mutant of ASXL1. In an alternative aspect, the cancer can be NSCLC and the target protein can be EML4-ALK.

### Method for Disrupting a Condensate in a Cell

In another aspect, disclosed herein is a method for disrupting a condensate in a cell, wherein the condensate comprises a concentrated population of a biomolecule, the method including at least the step of contacting the condensate with a fusion protein. In a further aspect, the fusion protein includes a J domain protein and a targeting molecule, and the targeting molecule binds the biomolecule. Further in this aspect, when the targeting molecule binds the biomolecule, the J domain protein recruits a chaperone protein to disassemble the condensate.

In one aspect, the biomolecule can be a target protein, although other biomolecules in addition to peptides and proteins are also contemplated as biomolecules and should be considered disclosed. In some aspects, the target protein can be a truncated mutant, although other types of mutations to wild type sequence and structure are also contemplated and should be considered disclosed. In some aspects, the target protein can include one or more intrinsically disordered regions.

Exemplary target proteins can include, but are not limited to, MLL-AF9, another MLL-fusion condensate, ASXL1, EML4-ALK, or any combination thereof. Exemplary chaperone proteins can include, but are not limited to, HSP70. In some aspects, the targeting molecule can be a nanobody or a target protein binding domain. In any of these aspects, the cell can be a mammalian cell.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a mutation,” “a blood cancer,” or “a condensate,” include, but are not limited to, mixtures, combinations, or groups of two or more such mutations, blood cancers, or condensates, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Establishing ASXL1 Condensation and Tumorigenicity

Molecular activity of ASXL1 ASXL1 -3, the three members of the mammalian ASXL family, are obligate factor for the BAP1 deubiquitinase to erase mono-ubiquitylation at lysine 119 of histone H2A (H2AK119ub), an epigenetic mark for gene repression. The catalytic activity of BAP1 requires its direct binding with the ASXH domain of the ASXLs, which stimulates BAP1 activity through stabilizing BAP1-ubiqutin binding. Though BAP1 is a tumor suppressor frequently mutated in solid tumors, it is rarely mutated in myeloid neoplasms. Rather, BAP1 with its deubiquitination activity promotes ASXL1 -associated myeloid malignancies. Compared to ASXL1 WT, ASXL1 leukemia truncations aberrantly enhance the deubiquitination effects of BAP1. In models using human hematopoietic stem and progenitor cells, mice, and primary leukemia cells from patients, BAP1 depletion or inhibition suppresses leukemogenesis or leukemia phenotypes, and inhibits expression and H2A deubiquitination of key leukemogenic genes. These studies highlight a critical role of BAP1 hyperactivity in ASXL1 -associated myeloid malignancies. While ASXL1 truncations may stabilize BAP1 and/or their binding, and modifications of ASXH domain can enhance ASXL1 stability, the different expression levels of WT and truncations and other factors often complicate these interpretations. It thus remains largely unclear how the ASXL1 truncating mutants uniquely confer BAP1 hyperactivity in H2A deubiquitination.

The frequent cancer truncations of ASXL1 , not WT, form condensates with transcriptional proteins. It has previously been shown that another chromatin modulator, UTX, suppresses cancer through forming phase-separated condensates mediated by its intrinsically disordered region (IDR), a property lost in its cancer truncation. Since ASXL1 has large IDRs throughout the protein and the most disordered region is lost in the most frequent cancer mutation, it was hypothesized ASXL1 might regulate cancer through condensation in a similar way to UTX. GFP-tagged ASXL1 WT and G646Wfs*12 and Y591X, the most frequent frameshift and nonsense mutations, respectively, were expressed in myeloid malignancies. G646Wfs*12 has an insertion of a nucleotide causing a frameshift that makes 12 amino acids before a stop codon. It was surprising to find that, exactly opposite to the present hypothesis, ASXL1 WT and (1-1067) appeared diffuse, whereas the two truncating mutants that lost large central and C-terminal IDRs formed foci in the nucleus. The immunostaining signals of endogenous ASXL1 showed discrete puncta in Kasumi-1 (ASXL1^{+/G646Wfs*12}) and primary blood cells of an AML patient (ASXL1^{+/G645fs}), but more diffuse in Jurkat (ASXL1^{+/+}) and ASXL1 WT AML patient cells, suggesting the endogenous truncations forming nuclear condensates. Purified ASXL1 1-403, 404-645, and 1-590 all formed liquid droplets that readily fuse. Surprisingly, the highly disordered 646-1067 did not form condensates under physiological conditions. 1068-1541 was insoluble and could not be readily purified.

Using CRISPR/Cas9, the N-terminus of the Y591X allele was tagged with FLAG-mEGFP (FmEGFP) in K562 cells (ASXL1^{+Y591x}). By genomic PCR, RT-PCR using GFP-ASXL1 primers and sequencing, and IPWestern, clones were identified where mEGFP was tagged at the Y591X allele. The endogenous FmEGFP-ASXL1^{Y591X} formed dynamic nuclear foci. Y591X foci significantly overlapped with proteins important for gene activation, including BAP1, BRD4, Ser2 phosphorylated RNA-Pol II, and WT ASXL1, but not with the heterochromatin protein HP1. (The incorporation of WT into the condensates of mutants may be relevant to the dominant effect of the mutations.) These results suggest that condensates of ASXL1 cancer mutants may act as hubs for active transcription in cancer cells.

Separation-of-function mutations of ASXL1. To rigorously show the function of condensation, it was sought to first identify key residues in the IDRs that specifically control condensation but not other activities, especially binding to BAP1 and BRD4, which are relevant to tumorigenesis. ASXL1 (1-645) IDRs have prominent blocks of oppositely charged residues, a pattern known to promote phase separation. Mutagenesis was thus started in the IDRs (not structured domains) of 1-590. Mutation of all 25 Arg in the IDRs to Ala (25RA) abolished 1-590 foci in cells. BAP1 binding is through the ASXH domain, while BRD4 binding is through an ASXL1 epitope (567-587). It was found 25RA did not affect BAP1 binding but disrupted BRD4 binding. Multi-rounds of mutagenesis were thus carried out in IDRs of (1-590). A mutant, 10RA, with 10Rs mutated to A in 391-426, was identified that reduced (but did not abolish) foci formation and still retained binding to BAP1 and BRD4. More quantitative analysis using purified proteins showed that 25RA substantially, and 10RA mildly, reduced condensation of 1-590.

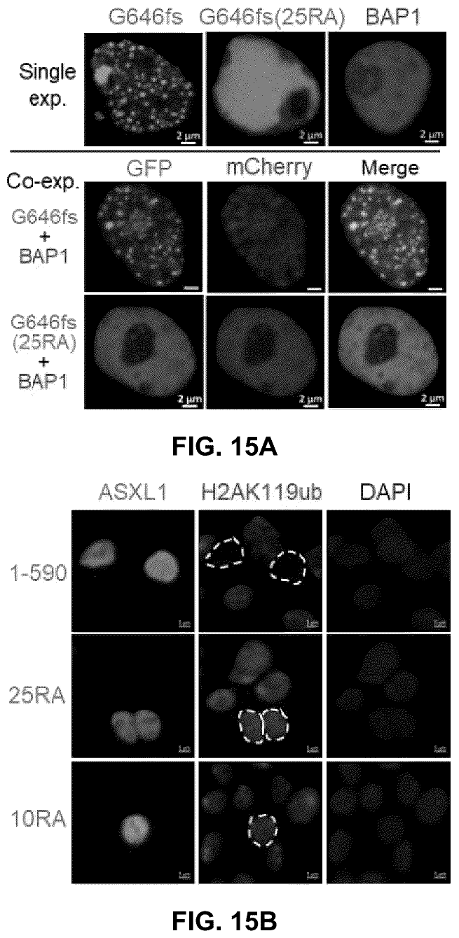

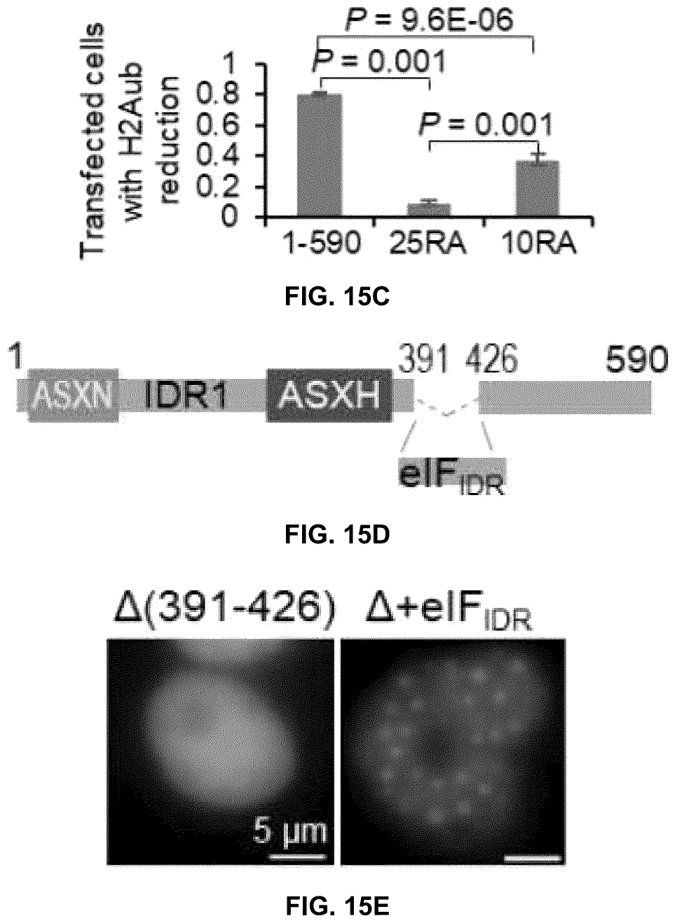

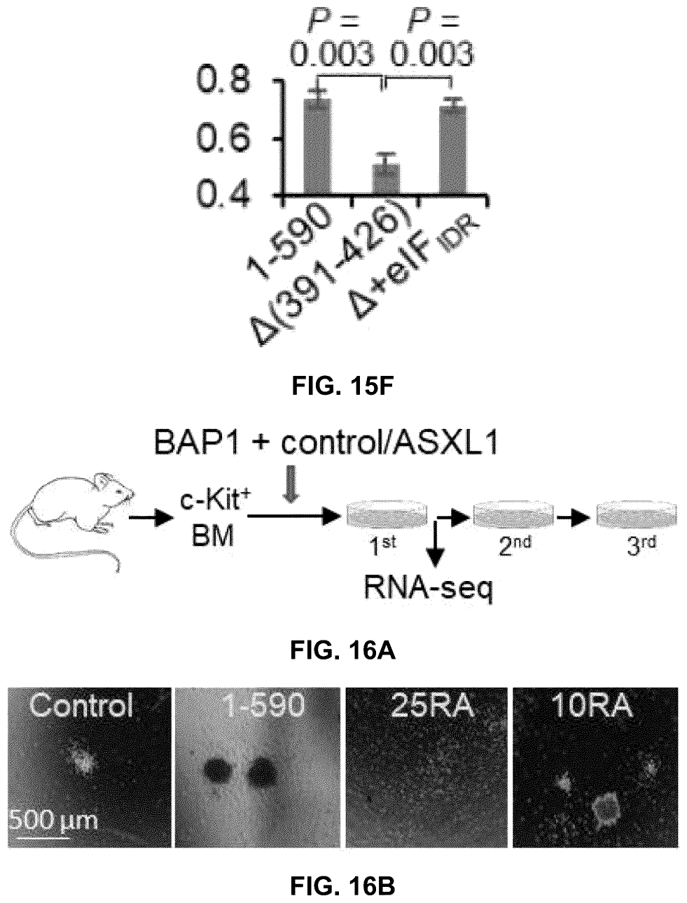

Condensates of ASXL1 truncations promote H2A deubiquitination by enriching BAP1 activity. How ASXL1 truncations confer BAP1 hyperactivity is unclear. This was reasoned to be is in part due to BAP1's enrichment in the ASXL1 truncation condensates: BAP1 by itself was diffuse in cells, but partitioned into the nuclear foci of the ASXL1 truncation, and remained diffuse when the truncation had 25RA (FIG. 15A). It was found that overexpression of ASXL1 (1-590) greatly reduced H2AK119ub levels, but 25RA nearly abolished, and 10RA significantly impaired, the ability of 1-590 in reducing H2AK119ub (FIGS. 15B-15C). Moreover, while deleting 391-426 (the minimal region for condensation found in these studies) from 1-590 impaired condensation and the ability to reduce cellular H2Aub, replacing it with the unrelated but condensate-forming IDR from a yeast translation initiation factor elF4GII restored condensation, and was sufficient to restore the deubiquitination activity (FIGS. 15D-15F). In the in vitro H2A deubiquitination assays with all purified proteins and ubH2A-nucleosome, it was found that 1-590, but not 25RA, enhanced BAP1 activity in deubiquitination. These results support ASXL1 truncation condensates enrich BAP1 to enhance H2A deubiquitination, which promotes myeloid malignancies.

Condensation of ASXL1 mutants promotes myeloid malignancies. Mutating endogenous Asxl1 in mice (to mimic human G646Wfs*12) has only very mild leukemia-related phenotypes after long latency, in accordance with the fact that ASXL1 mutations often occur in aged people and co-occur with other driver mutations. The field has established a few other robust functional assays by expressing these gain-of-function truncations in mouse bone marrow (BM), often together with BAP1 or another oncogene that co-occurs in patients. Based on these methods, three assays were used to study condensation of ASXL1 truncations in promoting myeloid cancer, which also forms the main functional assays for the studies.

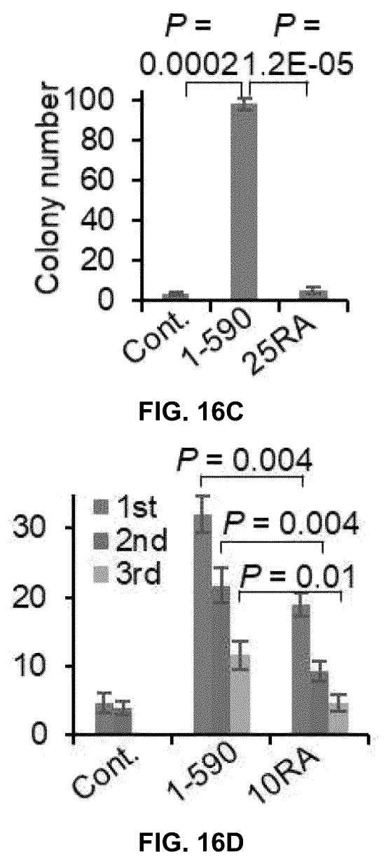

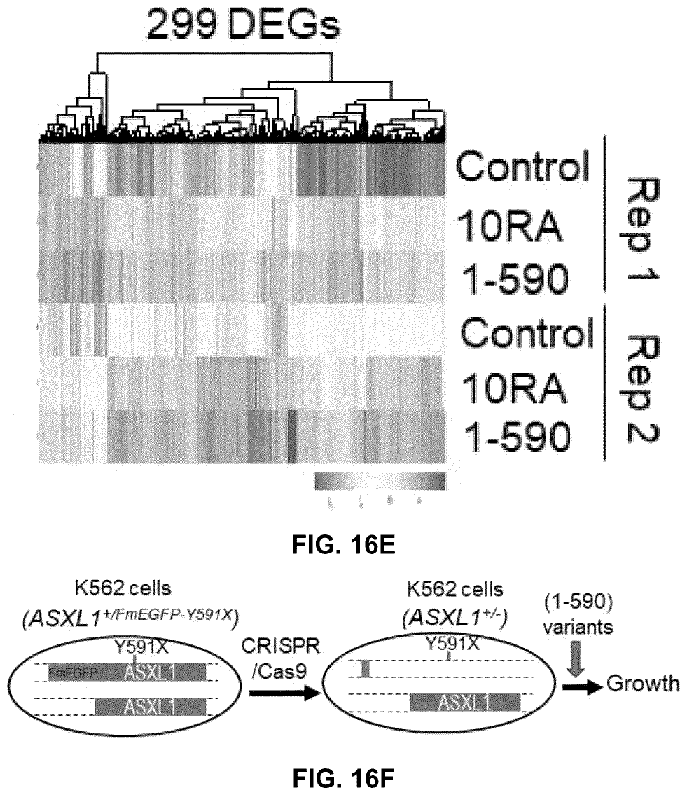

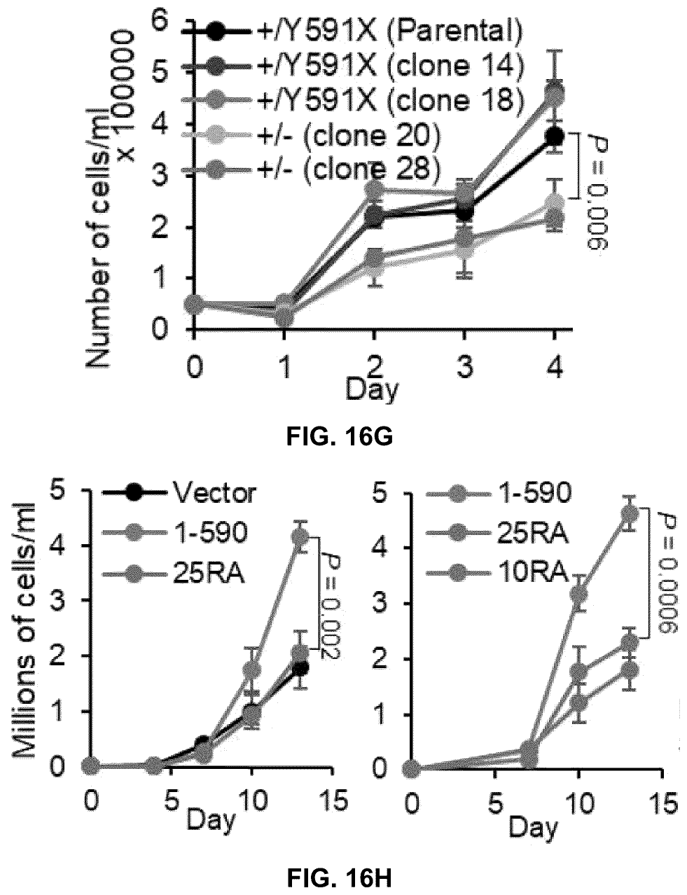

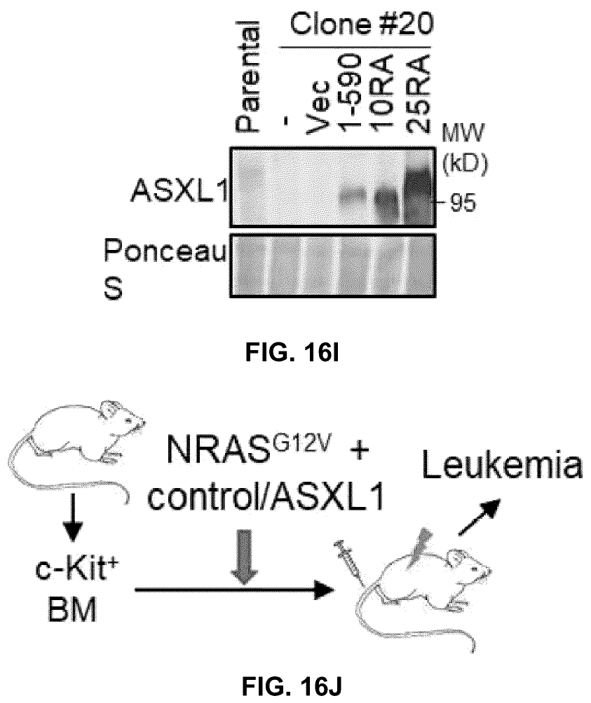

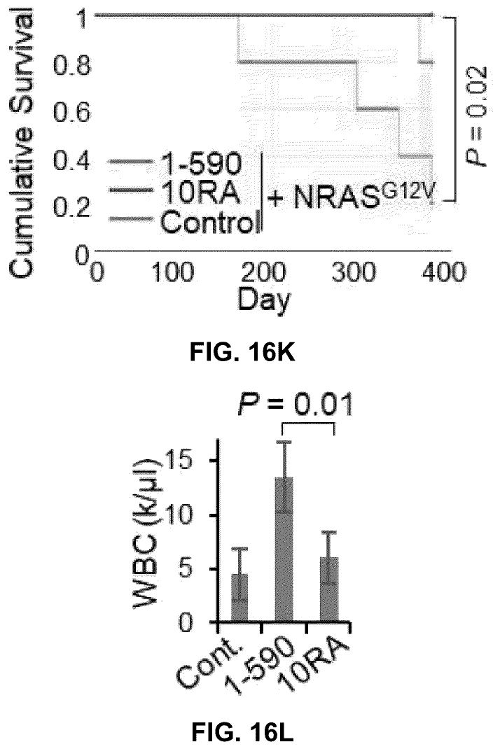

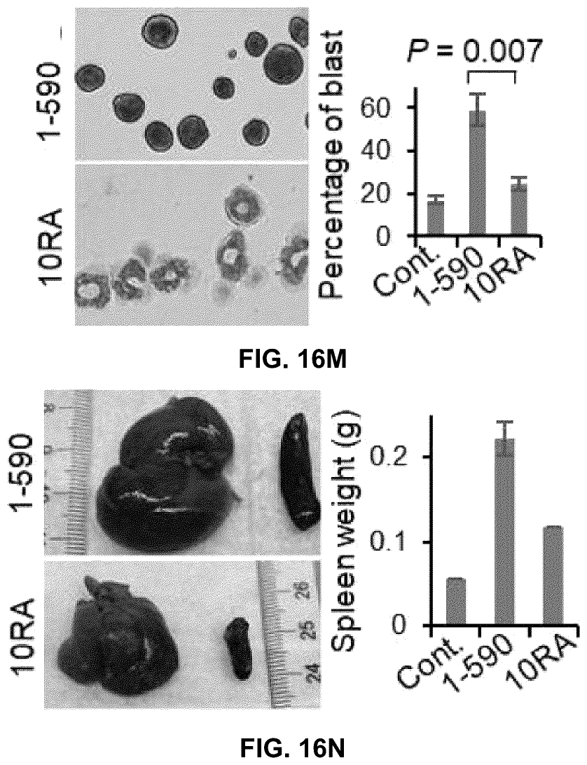

The first assay. Mouse c-Kit+BM cells were co-transduced with BAP1 and a control vector, ASXL1 (1-590), or 1-590 (25RA or 10RA), and performed colony formation assays (FIG. 16A). Colony formation was significantly enhanced by 1-590, but abolished by 25RA and significantly impaired by 10RA (FIGS. 16B-16D). Genes that were upregulated by co-transduction of BAP1 and 1-590 but less efficiently by BAP1 and 10RA were significantly enriched with myeloid lineages, leukocyte migration, and inflammatory/innate immune response (FIG. 16E). Since inflammation is an important mediator in ASXL1 mutation-associated myeloid malignancies and clonal hematopoiesis, these results suggest that 1-590 condensation is important in promoting leukemogenesis-associated colony formation through efficient activation of myeloid tumorigenic transcriptional programs.

The second assay. Deletion of ASXL1^{Y591X} in K562 cells resulted in reduced growth and increased global level of H2AK119ub. CRISPR-deletion of ASXL1^{Y591X} in K562 cells (resulting in ASXL1^{+/-}) (FIG. 16F) also reduced growth (FIG. 16G). This was rescued by transduction of (1-590), but not efficiently by (1-590) 25RA or 10RA (FIGS. 16H-16I), suggesting that condensation of Y591X promotes the growth of myeloid leukemia cells.

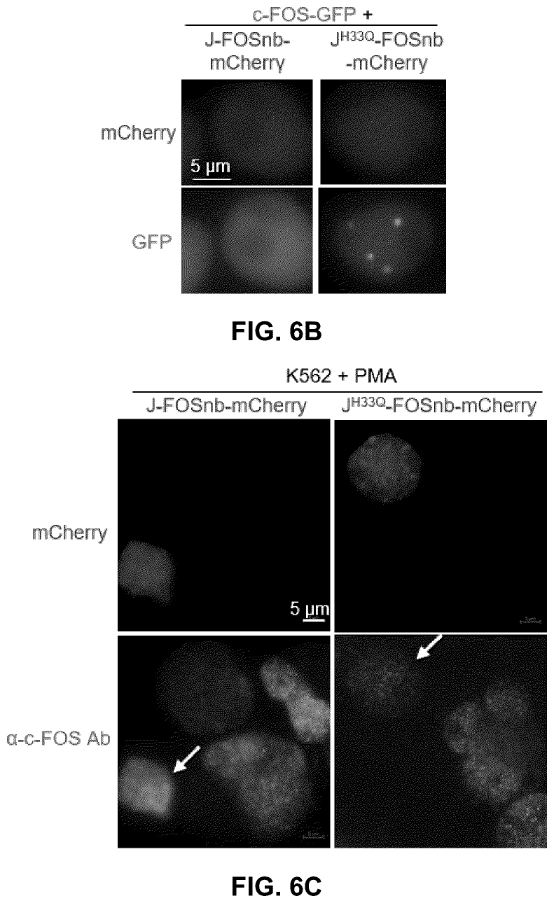

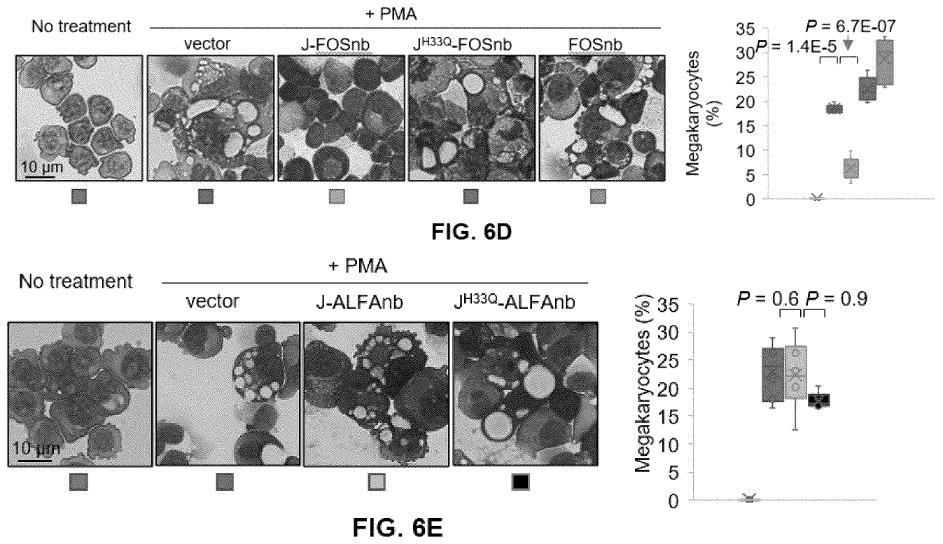

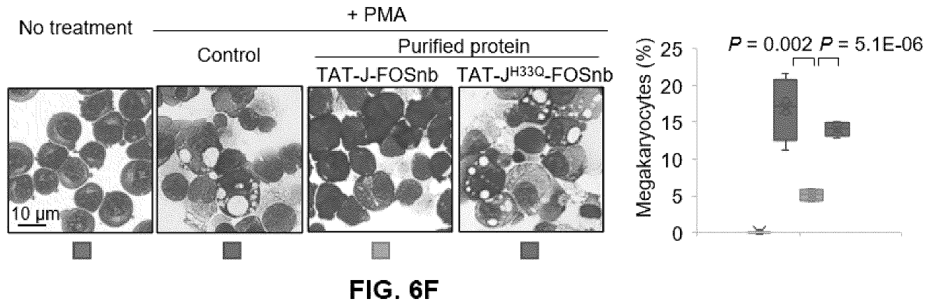

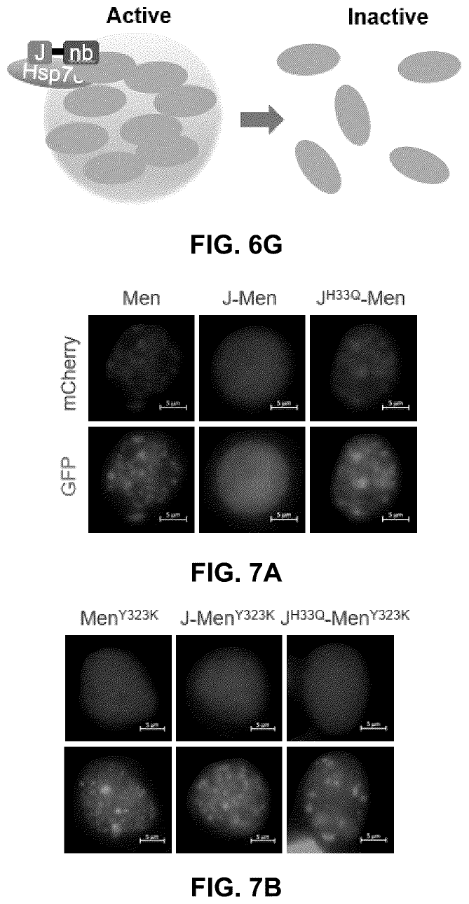

The third assay. ASXL1 mutations frequently co-occur with NRAS mutations in human myeloid malignancies. Mouse c-Kit+ BM was transduced with NRASG12V and control vector, ASXL1 (1-590), or 1-590 (10RA), and transplant the cells to irradiated recipient mice (FIG. 16J). Co-expression of 1-590 with NRAS^{G12V} significantly accelerated leukemogenesis and shortened survival in the recipients than NRAS^{G12V} alone, but co-expression of 10RA had a significantly less effect on promoting leukemogenesis, with improved survival compared to 1-590 (FIG. 16K). This is shown by the enhanced white blood cell count with greatly enhanced number of immature blast cells in the myeloid lineage and hepatosplenomegaly in mice transplanted with cells co-expressing 1-590, but much less of any of these leukemic phenotypes in mice with 10RA (FIGS. 16L-6O). Taken together, these data suggest that the condensation property in the ASXL1 N-terminal region is crucial for the truncations to promote myeloid malignancies through facilitating BAP1-mediated H2A deubiquitination and transcriptional activation of myeloid leukemogenic pathways.

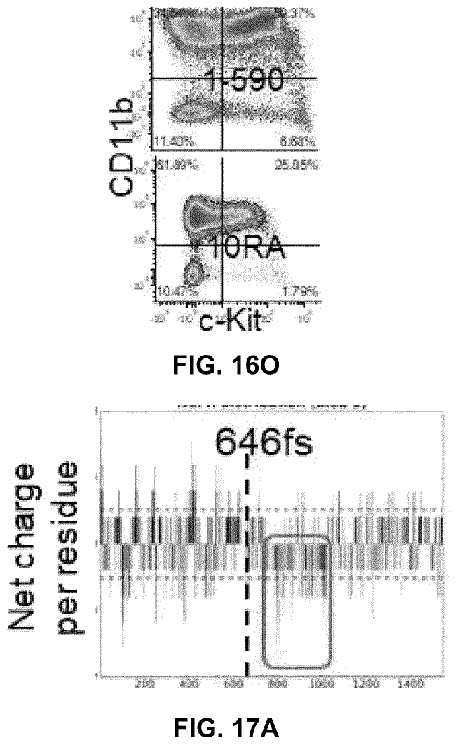

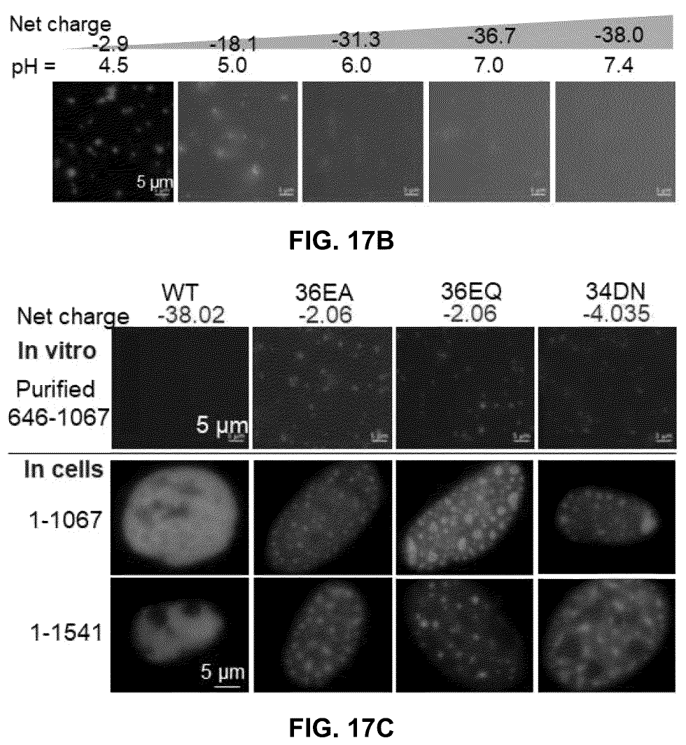

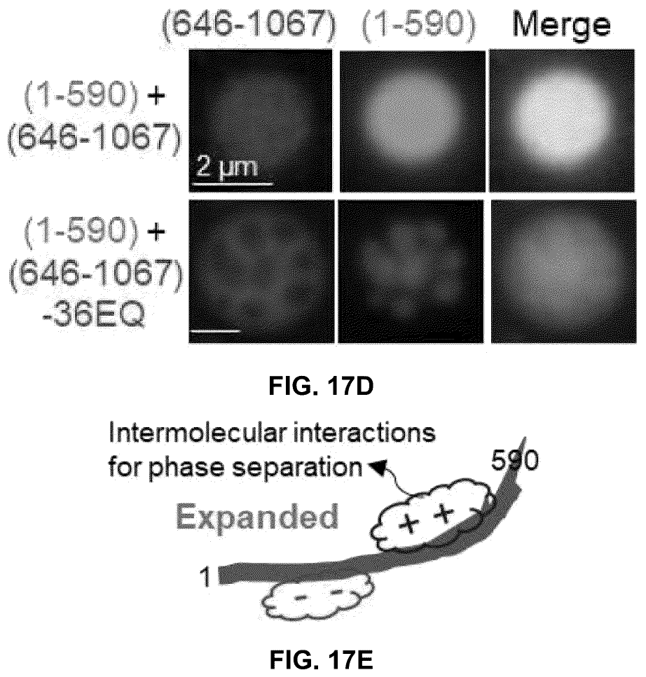

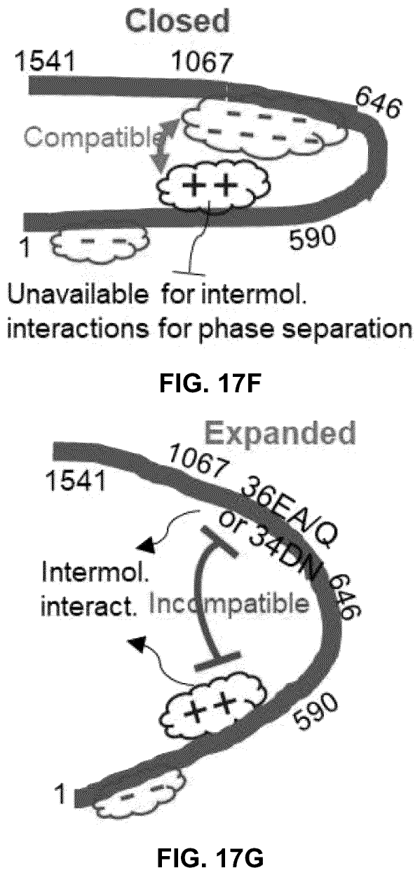

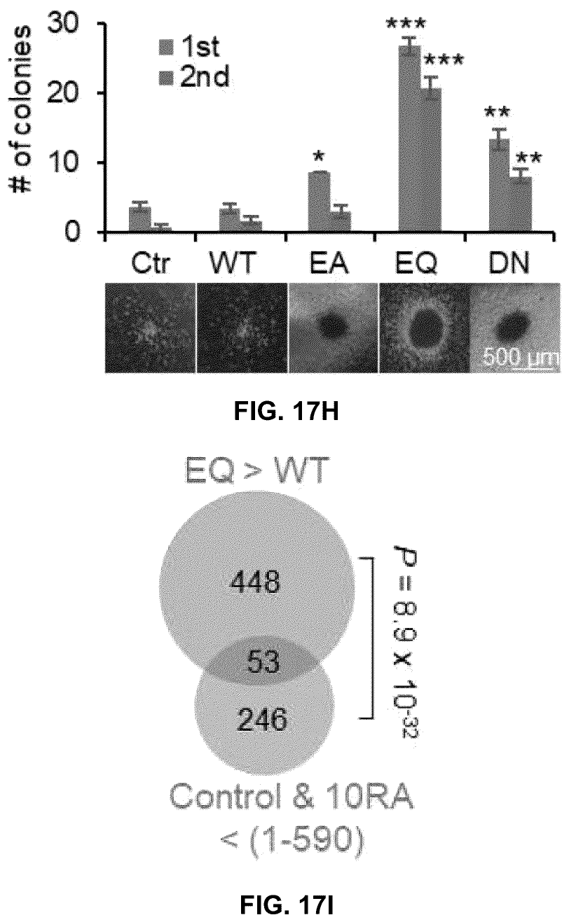

The frequently deleted region regulates ASXL1 condensation and tumorigenicity. It was next sought to determine the mechanisms and significance for the suppression of condensation by the frequently deleted region. It was found that 646-1067 has a prominent feature of high net negative charge (FIG. 17A) that is conserved throughout evolution (not shown). Purified 646-1067 did not form major condensates at pH 7.4, but restored condensation as pH was reduced to 4.5 and the net charge was abolished (FIG. 17B). All 36 Glu were then mutated to Ala (36EA) or Gln (36EQ), or all 34 Asp to Asn (34DN) in 646-1067 to restore charge neutrality. All these mutations restored condensation of purified 646-1067 at physiological pH levels, and also condensation of both 1-1067 and full-length (1-1541) expressed at levels similar to the WT counterparts in cells (FIG. 17C). Therefore, the negative charge of the frequently deleted region suppresses ASXL1 condensation in physiological conditions.

While purified 646-1067 itself does not form condensates at physiological pH levels, it formed co-condensates with the co-incubated 1-590, and was uniformly incorporated into the condensates (FIG. 17D), suggesting attractive interactions between these two regions. 646-1067 with 36EA/Q or 34DN mutations, however, formed condensates that occupied mutually exclusive space with 1-590, indicating incompatible or repulsive interactions between them (FIG. 17D). Therefore, it is reasoned that the attractive interactions between the positive charge of 1-590 and the negative charge of 646-1067 make the former unavailable for intermolecular interactions required for condensation. The frequent truncations unleash the positive charge of 1-590 to be engaged in intermolecular interactions for condensation. Charge-neutralizing mutations in 646-1067 lose attractive interactions or gain repulsive interactions with the N-terminal regions, thereby making the latter available for intermolecular interactions driving phase-separation (FIGS. 17E-17G).

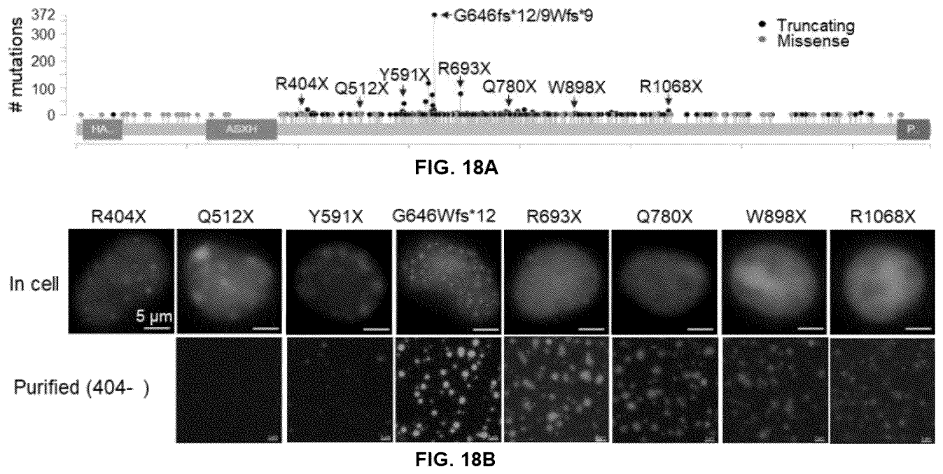

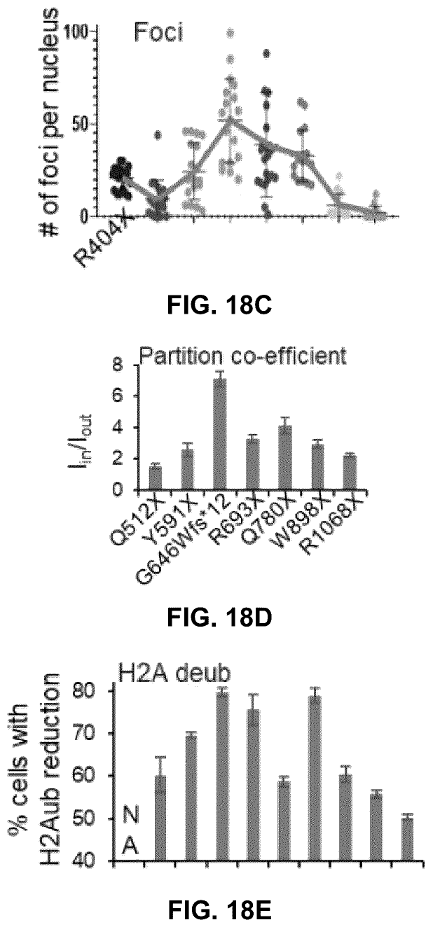

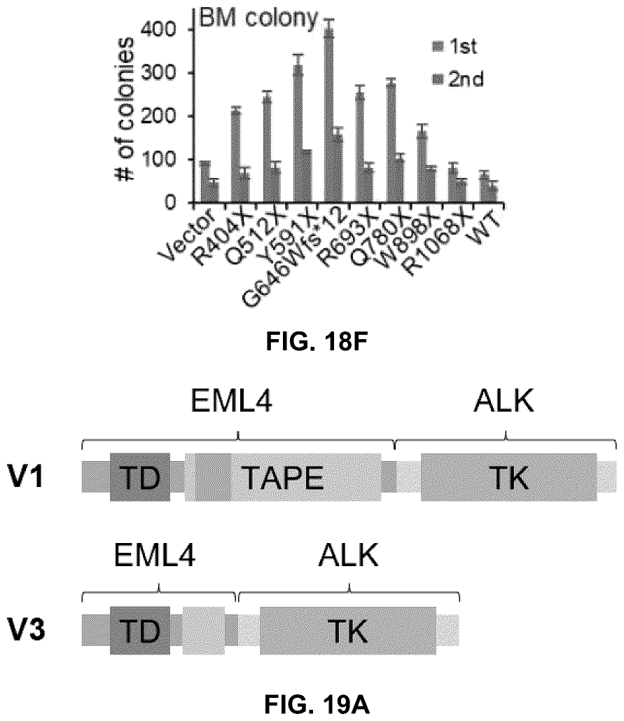

On the function level, while ASXL1 (1-1067) was inactive in driving BM colony formation, the 36EA, 36EQ, or 34DN mutations in 646-1067 rendered 1-1067 active in colony formation (FIG. 17H) (tests on the full-length ASXL1 were precluded by their poor transduction/expression in BM), and also enhanced the ability of 1-1067 in inducing genes enriched for myeloid development and innate immune response (FIG. 17I). These induced genes significantly overlap with the genes that were activated by 1-590 but less efficiently by 1-590 (10RA) (FIG. 17I), suggesting that the charge-neutralizing and condensation-restoring mutations turned the non-clonogenic 1-1067 clonogenic in part through inducing transcriptional programs elicited by condensates of cancer truncations. Moreover, a series of ASXL1 myeloid malignancies patient mutations from 404 through 1068 were analyzed, and it was found that their condensation abilities well correlate with their leukemia-promoting activities including H2A deubiquitination and BM clonogenesis (FIGS. 18A-18F). These results strongly suggest that dysregulated condensation is a central mechanism in driving tumorigenicity of ASXL1 IDR mutations.

From mechanisms to potential intervention. Having shown that ASXL1 truncating mutants promote blood cancer through forming condensates, it was sought to develop a strategy to disrupt these condensates to inhibit the associated blood cancer based on two rationales: (i) Molecular chaperones, most prominently Hsp70s, actively regulate the formation and properties of phase-separated condensates in the cell. J-domain proteins (JDPs) are co-chaperones for Hsp70s and function to target the substrates to Hsp70s (via J domain-Hsp70 binding) and activate Hsp70 to disperse target aggregation and condensation. (ii) J-domain fusion to the catalytic subunit of protein kinase A (PKAcat) dissolves PKA condensates and causes human cancer PKA condensation is unaffected by fusion to J^{H33Q}, a mutation in J-domain that abolishes Hsp70 activation. ASXL1 mutants are thus targeted to Hsp70s for condensate disruption. Preliminary data below show that fusion of J-domain with a number of different condensate-forming proteins, including an ASXL1 mutant, not only dissolved the condensates in cells, but also disrupted the biological functions of the condensates.

### Example 2: Results

### J-Domain Fusion to Different Proteins Leads to Condensate Dispersal and Functional Disruption

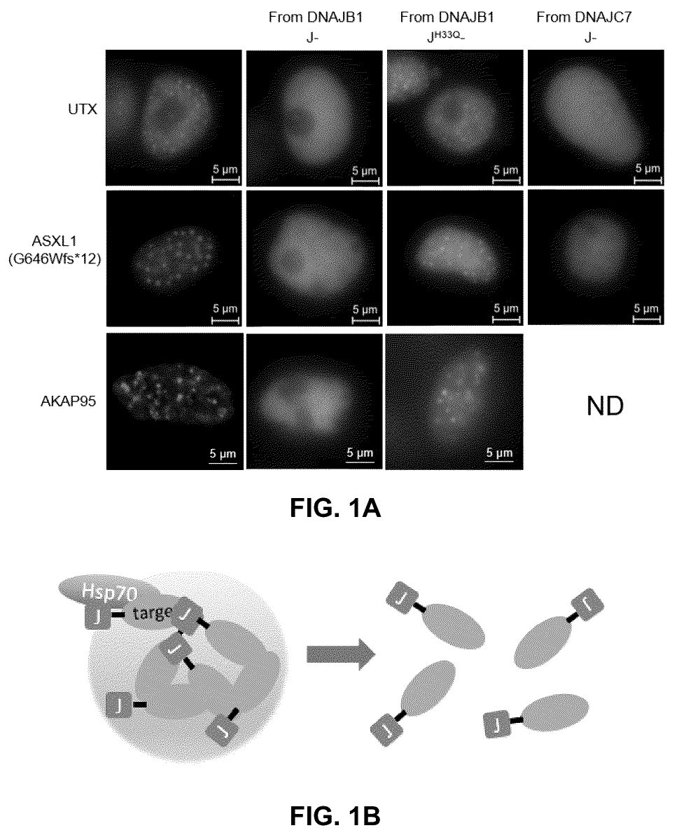

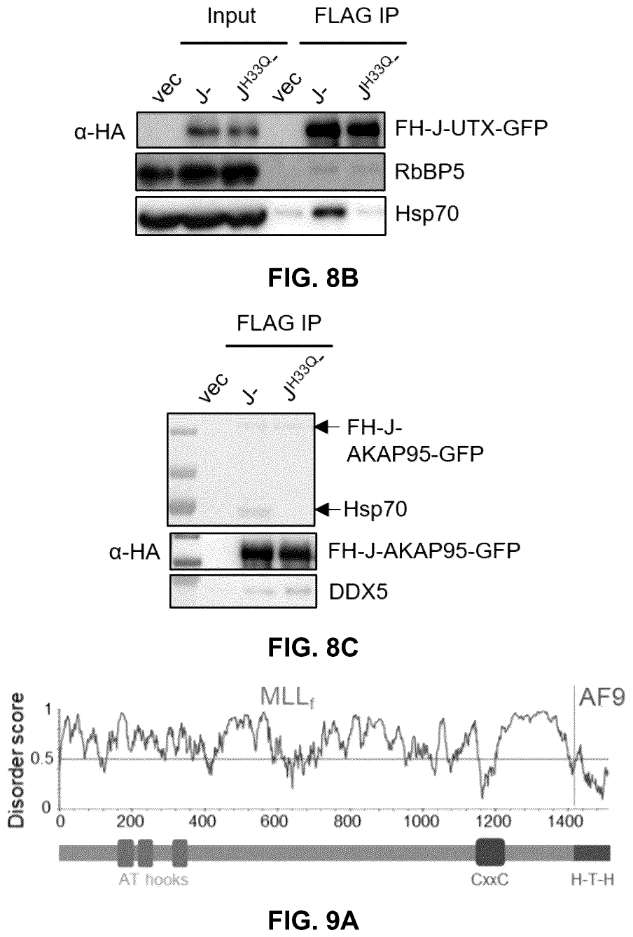

DNAJB1 J domain or J^{H33Q} mutant was first fused to a number of proteins that have been shown to form condensates, including AKAP95, UTX, and ASXL1^{G646Wfs*12} (the most frequent mutation, a truncation, in myeloid malignancies, see the accompanying manuscript). It was found that J-domain fusion to any of these proteins abrogated condensate formation without affecting overall protein levels as shown by the signal intensities. Conversely, fusion to J^{H33Q} did not reduce condensate formation (FIG. 1A). Moreover, the J domain from DNAJC7 also showed similar effect on these condensates (FIG. 1A). DNAJB1 J domain was thus used for the rest of this study. Quantitative analysis of the images showed that J-domain fused target proteins, as compared to unfused or J^{H33Q}-fused target proteins, did not affect GFP signal intensity (FIG. 8A), suggesting that the recruited Hsp70 is unlikely to unfold GFP (since GFP needs to be correctly folded to be fluorescent), though it is less clear if it affects fold of the target protein. Co-immunoprecipitation (co-IP) assays showed that the J-domain fusions bound to Hsp70, while the J^{H33Q} variants did not, indicating that the effects of the J-fusion on condensates require Hsp70 recruitment and activity (FIGS. 8B-8C). Importantly, J-domain fusion did not affect interaction of the target proteins with their normal binding proteins, as shown by the comparable amount of RbBP5 (a core subunit of the MLL3/MLL4 complexes) associated with UTX fused to J and J^{H33Q}, as well as DDX5 (also known as p68) associated with AKAP95 fused to J and J^{H33Q} (FIGS. 8B-8C).

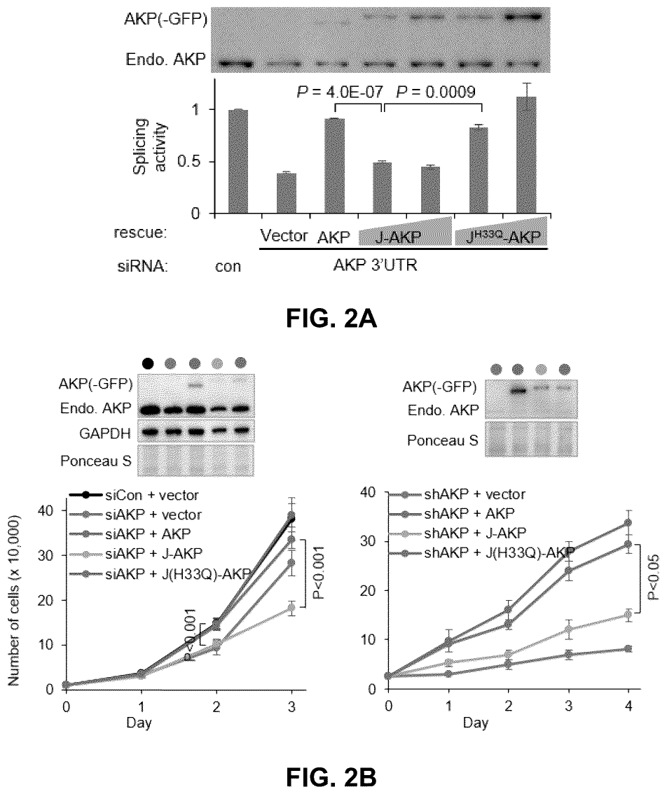

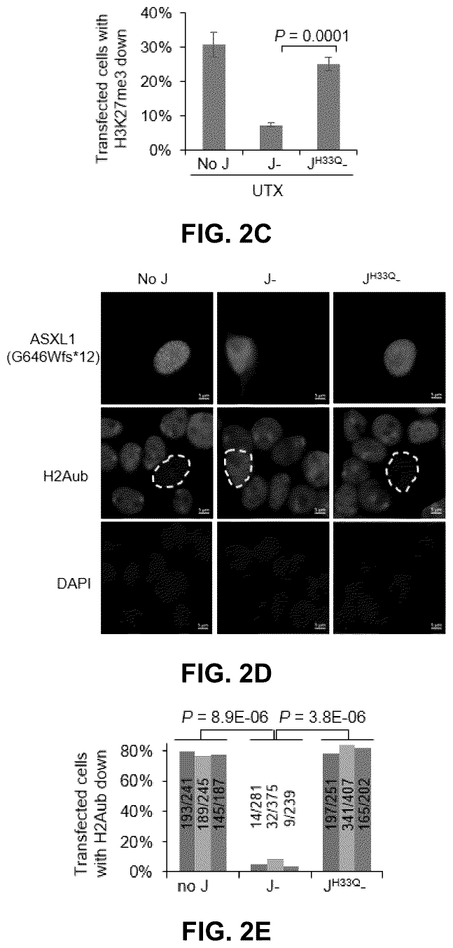

Various condensate-dependent activities were next assessed. It was previously shown that reporter splicing and cancer cell growth were reduced by AKAP95 depletion via siRNAs targeting the endogenous 3′ UTR. This effect could be rescued by expression of WT AKAP95 lacking a 3′ UTR (FIG. 2A). It was found that expression of J-AKAP95 failed to rescue splicing, whereas J^{H33Q}-AKAP95 rescued splicing in a dose-dependent manner at near or sub-endogenous levels (FIG. 2A). Moreover, growth of AKAP95-depleted cancer cells was rescued by J^{H33Q}-AKAP95, but not by J-AKAP95 (FIG. 2B). It was previously shown that condensation of UTX facilitates H3K27 demethylation in cells. Here it was found that UTX fusion to J, but not to J^{H33Q}, greatly reduced its H3K27 demethylation activity in cells (FIG. 2C). This is consistent with J-UTX, but not J^{H33Q}-UTX, disrupt UTX condensates and the associated demethylation activity. The effects on ASXL1^{G646Wfs*12} were also tested, since this forms nuclear condensates to facilitate H2A deubiquitination that is associated with myeloid cancer. J-ASXL1^{G646Wfs*12} almost completely lost the activity in H2A deubiquitination, while J^{H33Q}-fusion had no effect (FIGS. 2D-2E), suggesting that dispersing the ASXL1^{G646Wfs*12} condensates disrupt its associated biochemical activity.

Taken together, these results demonstrate that recruiting Hsp70 activity by J-domain fusion can disperse condensates and disrupt the condensate-associated biological functions (FIG. 1B).

### Targeting Condensates to Chaperones by Nanobodies Disperses Condensates and Disrupts Function

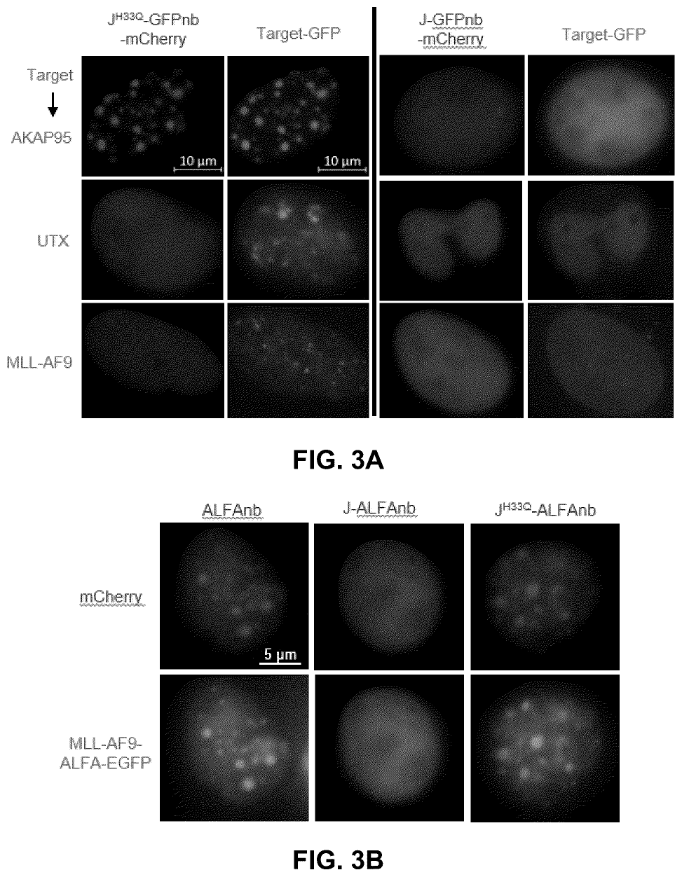

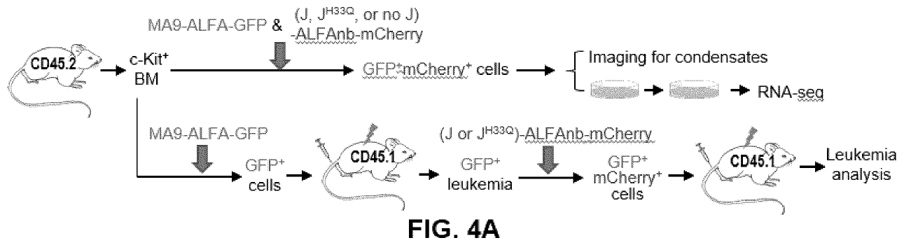

Direct fusion of J-domain to a target protein would not be convenient for studying the function of the endogenous protein condensates and also not a viable therapeutic approach. It was thus sought to recruit Hsp70s to the target proteins by linking the J-domain with a nanobody (nb), a small (˜15 kDa) single heavy chain variable domain from Camelidae. A GFP targeting nanobody (GFPnb) was fused with an HA epitope tag to either -J or J^{H33Q} and mCherry in a dox-inducible lentivirus. GFP-tagged UTX and AKAP95 all formed abundant condensates in cells co-expressing J^{H33Q}-GFPnb, while condensate formation was reduced in cells expressing J-GFPnb (FIG. 3A). These data suggest J-nb recruited Hsp70s to the tagged targets to disassemble the condensates.

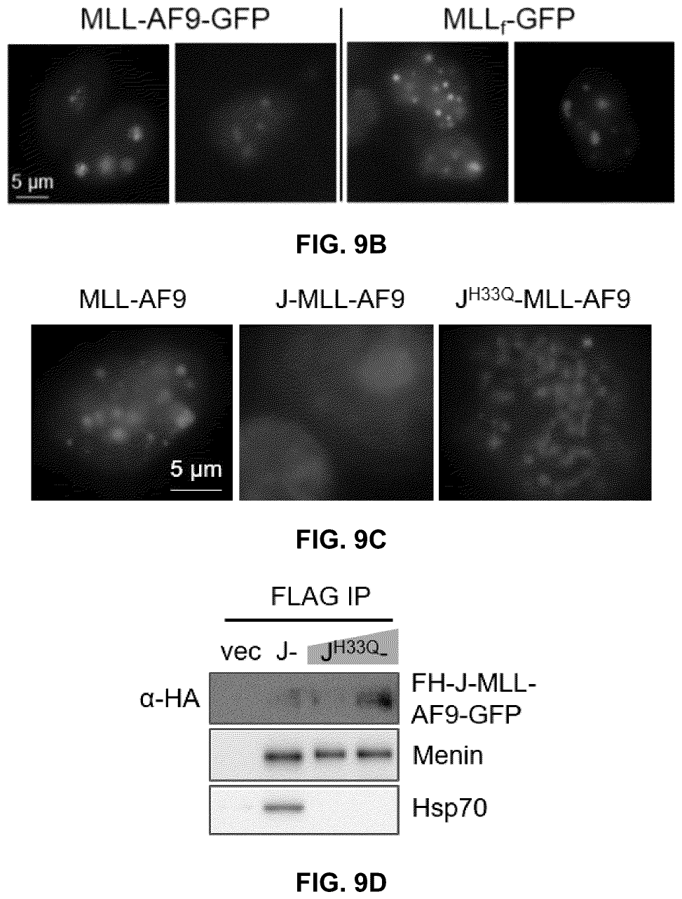

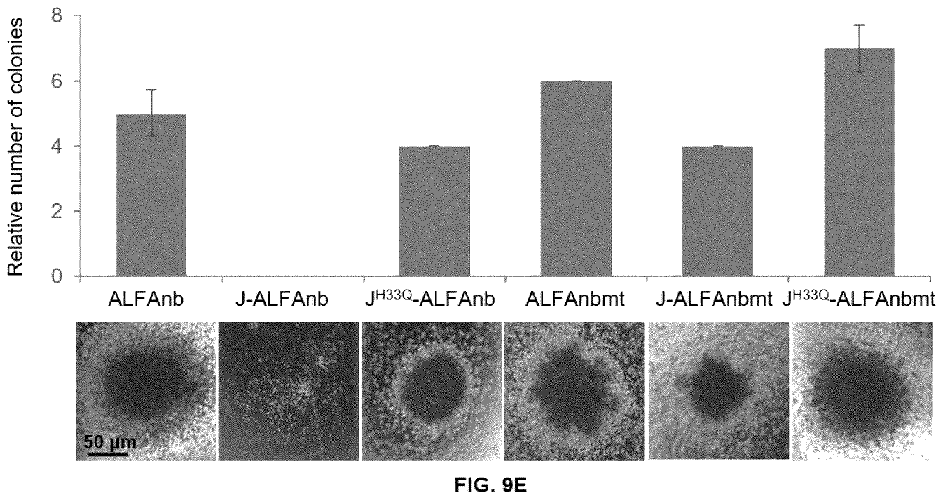

As the data above have demonstrated the effects of chaperone recruitment on proteins known to form condensates whose biological functions have been experimentally demonstrated (mostly through mutational studies), it was next sought to use this approach to particularly study proteins that are well known to drive important biological or pathological processes but have not been reported to form functionally relevant condensates. To this end, MLL-AF9, a fusion protein that results from translocation of MLL to AF9 and is a potent leukemia driver responsible for a high percentage of pediatric leukemias, was focused on. The MLL fusion proteins have been shown to form nuclear puncta or condensates, but it is unknown whether these condensates have any function, especially in driving leukemia. It was found that both MLL-AF9 and the MLL portion of the fusion (MLL_{f}) form nuclear condensates (FIGS. 3A and 9B), suggesting that MLL_{f}, with its large and extensive IDRs (FIG. 9A), is the main driver of the MLL-AF9 condensates. Fusion with the J-domain, but not with J^{H33Q}, effectively dispersed the MLL-AF9 condensates (FIG. 9C), indicating that MLL-AF9 condensates can also be regulated recruited chaperones. J-domain fusion did not affect the binding of MLL-AF9 with Menin (FIG. 9D), a well-established binding partner of MLL that is important for MLL-AF9-driven leukemogenesis.

It was then sought to study the effects of targeting MLL-AF9 condensates to molecular chaperones on the condensate formation and MLL-AF9 in leukemia function. It was found that condensates of the GFP-tagged MLL-AF9 were dispersed in cells expressing J-GFPnb but not J^{H33Q}-GFPnb (FIG. 3A). MLL-AF9 tagged with GFP and the ALFA peptide sequence (14-15 aa) using J domain or J^{H33Q}-fused to a nanobody against ALFA (ALFAnb) was also tried, which has a picomolar affinity for the ALFA tag, and got the same results (FIG. 3B). To determine if the condensate-dispersing effect requires specific binding with the target protein, an ALFAnb mutant (ALFAnbmt) was designed that contains E58A, R59A, N61A, R65E, and D112K mutations, which should disrupt the interaction between ALFAnb and the ALFA peptide based on the co-crystal structure of ALFA-ALFAnb complex. In contrast to ALFAnb, the ALFAnb mutant did not co-localize with the ALFA-GFP-tagged MLL-AF9 (FIG. 3B), suggesting that binding of this mutated nb with the ALFA sequence was lost. Fusion of neither J nor J^{H33Q} with this mutated ALFAnb had any effect on the condensates of the ALFA-GFP-tagged MLL-AF9 (FIG. 3B), indicating that the condensate-dispersing effect is dependent on the specific binding of the J domain-fusion construct with the target protein.

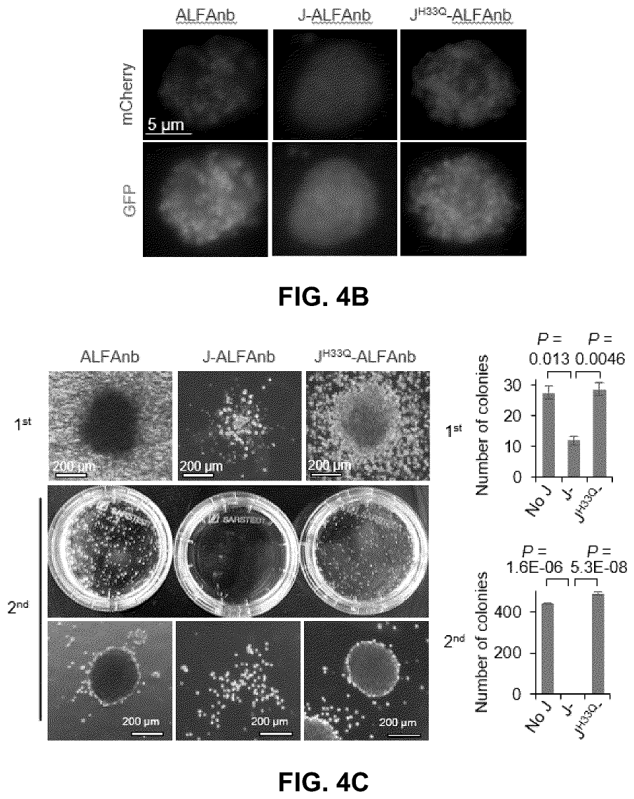

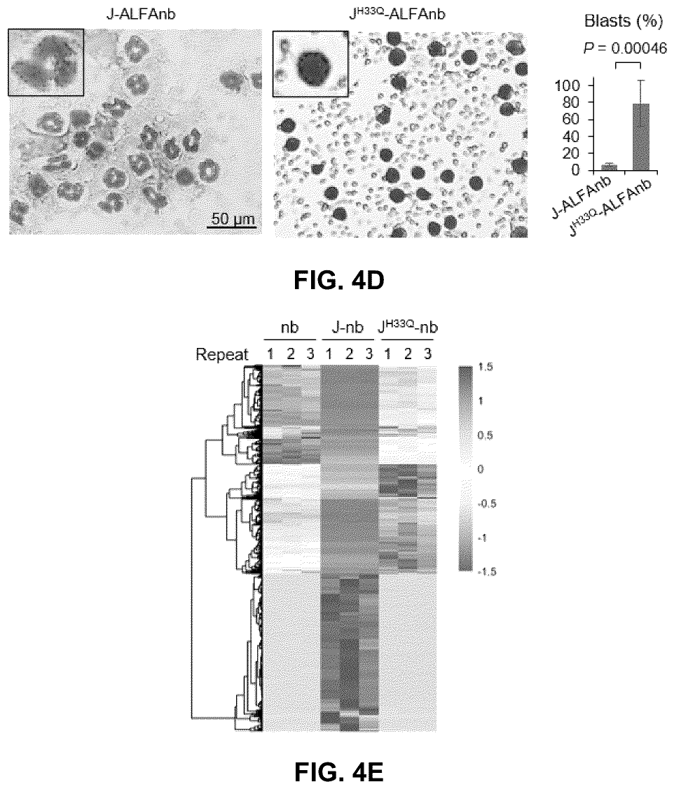

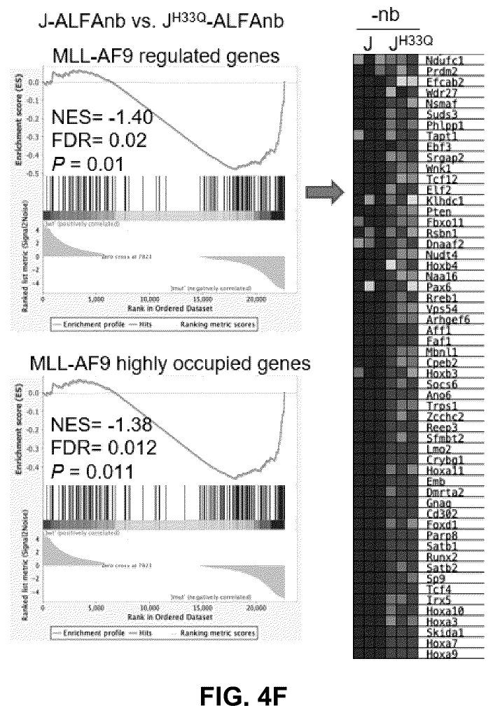

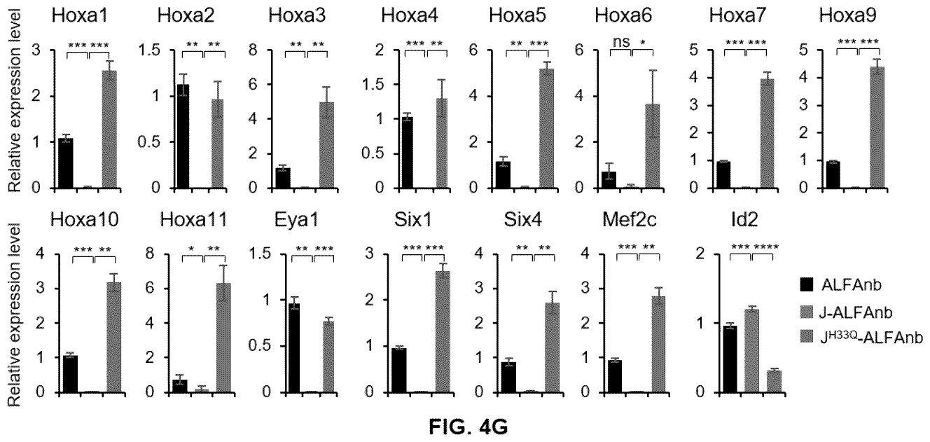

It was then investigated whether this strategy disrupts the potent leukemogenicity of the MLL-AF9 fusion using well-established assays including viral-transduction of mouse bone marrow (BM) followed by colony formation and transplant. Mouse c-Kit⁺ bone marrow was co-transduced with the ALFA-GFP-tagged MLL-AF9 and (no J, J, or J^{H33Q})-nb-mCherry viruses, sorted the GFP⁺mCherry⁺ cells for colony formation assays (FIG. 4A). MLL-AF9-ALFA condensates in the bone marrow cells were efficiently dispersed by J-ALFAnb, but not J^{H33Q}-ALFAnb (FIG. 4B). Strikingly, the activity of MLL-AF9 in driving the serial replating of bone marrow in colony formation was completely abolished by J-ALFAnb, but unaffected by J^{H33Q}-ALFAnb (FIG. 4C). Importantly, bone marrow colony formation was not affected by either unfused or J- or J^{H33Q}-fused ALFAnb mutant that failed to bind to ALFA and disperse the ALFA-tagged MLL-AF9 condensates (FIG. 9E), indicating that the biological effects were caused by targeting the specific target protein condensates, rather than a non-specific effect of overexpressing a protein that could potentially affect molecular chaperones and overall protein homeostasis in the cell.

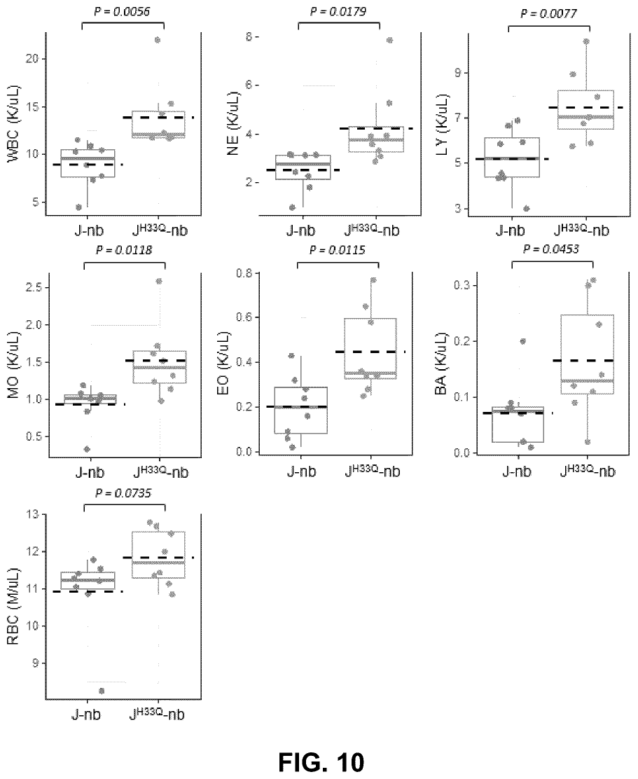

To determine if dispersing the MLL-AF9 condensates through chaperone recruitment can inhibit the development of pre-established leukemia in vivo, a mouse leukemia mode was established by transplant of mouse bone marrow transduced with the ALFA-GFP-tagged MLL-AF9. Bone marrow was then derived from the moribund, leukemic mice, transduced with J-ALFAnb or J^{H33Q}-ALFAnb, and performed secondary transplant to recipient mice (FIG. 4A). Although death of most mice has not yet been observed (except that one mouse that received J^{H33Q}-ALFAnb-transduced BM died of leukemia with enlarged liver and spleen) likely due to the inefficient viral transduction and limited cell number used in transduction, clear signs of leukemogenesis in J^{H33Q}-ALFAnb but not J-ALFAnb-transduced mice are present. It was found that the periphery blood of the mice transplanted with the J^{H33Q}-ALFAnb-expressing bone marrow was comprised mostly of cells with blast-like morphology, whereas that with J-ALFAnb-expressing bone marrow had nearly no cells with such morphology but was comprised mostly of cells that showed profound morphologic changes consistent with terminal differentiation (FIG. 4D). J-ALFAnb transduction significantly lowered the counts of all types of white blood cells, but not the red blood cells, in the recipient mice compared to transduction of J^{H33Q}-ALFAnb (FIG. 10). These results indicate that J-ALFAnb reduced the leukemia burden driven by the ALFA-tagged MLL-AF9 in vivo.

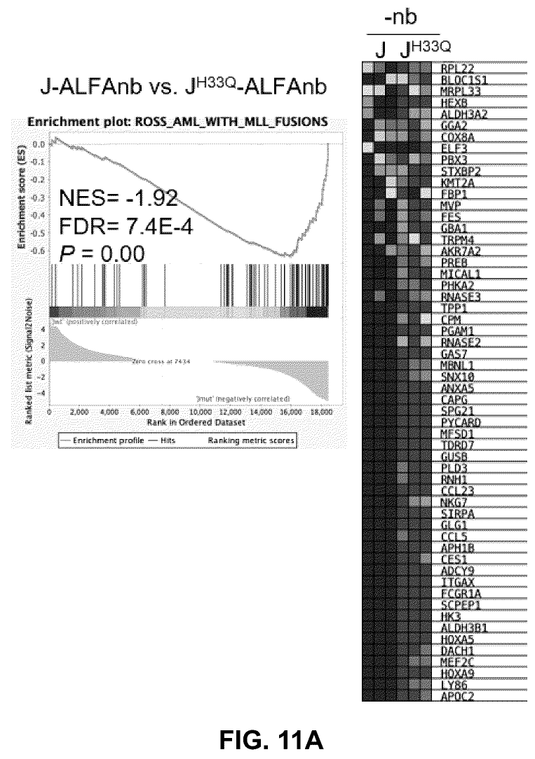

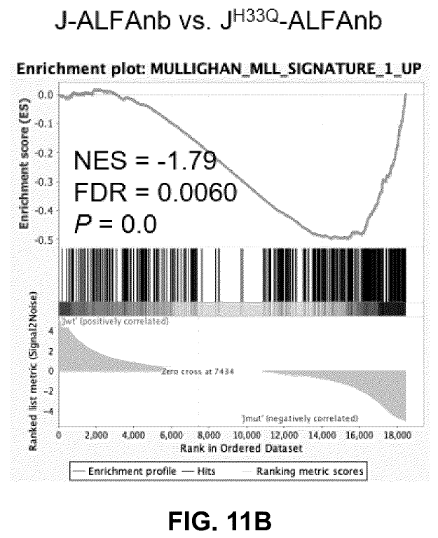

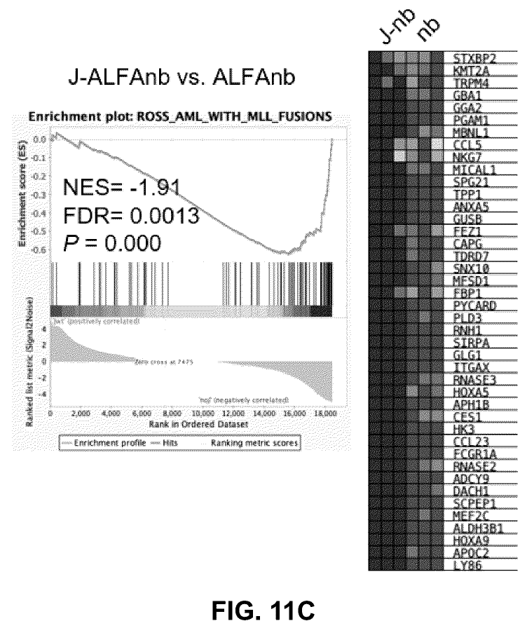

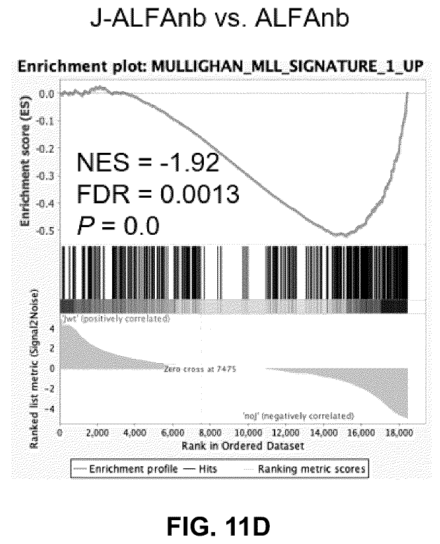

As MLL-fusions are known to drive leukemogenesis through transcriptional dysregulation, especially aberrant activation of many potent leukemogenic genes, the effects of J-ALFAnb on the MLL-AF9-driven transcriptional programs were assessed through RNA-seq analysis. J^{H33Q}-ALFAnb had little effect on the global gene expression programs compared to ALFAnb, whereas J-ALFAnb strongly changed the gene expression compared to both the ALFAnb and J^{H33Q}-ALFAnb in the MLL-AF9-ALFA-transduced bone marrow (FIG. 4E). Genes downregulated by J-ALFAnb were significantly enriched with the MLL-AF9 target genes defined either functionally or physically (MLL-AF9 regulated genes and MLL-AF9 highly occupied genes, respectively), and genes overexpressed in MLL-rearranged pediatric acute myeloid leukemias from two different resources (FIGS. 4F and 11A-11D). In particular, J-ALFAnb expression dramatically downregulated the expression of many well-established MLL-fusion target genes crucial in driving leukemogenesis including many of the Hoxa genes, Eya1, Six1, and Six4 (FIG. 4G). MLL-AF9 also drives leukemogenesis by suppressing the expression of certain genes such as ID2, which is an inhibitor of MLL-AF9-driven leukemogenesis through antagonizing the self-renewal and promoting differentiation of the leukemia stem cells. J-ALFAnb upregulated the expression of ID2 compared to ALFAnb and J^{H33Q}-ALFAnb in the MLL-AF9-ALFA-transduced bone marrow (FIG. 4G). These results support that MLL-AF9 condensation is crucial for the oncogenic transcriptional activity of MLL-AF9 and the present condensate-dispersing strategy effectively counteracts the leukemogenicity of MLL-AF9 through disrupting its transcriptional function.

Therefore, these data indicate that targeting different condensate-forming proteins to Hsp70s through J-domain fusion not only disperses the condensates but also disrupts the condensate-dependent biological function, and thus is likely broadly applicable to other protein condensates.

### A Direct Role of Hsp70 Chaperoning Activity on Dispersing Specific Condensates

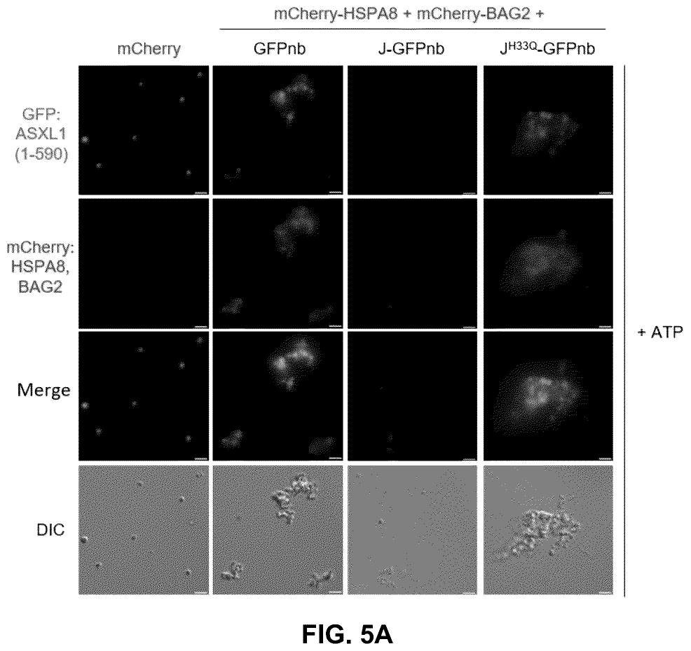

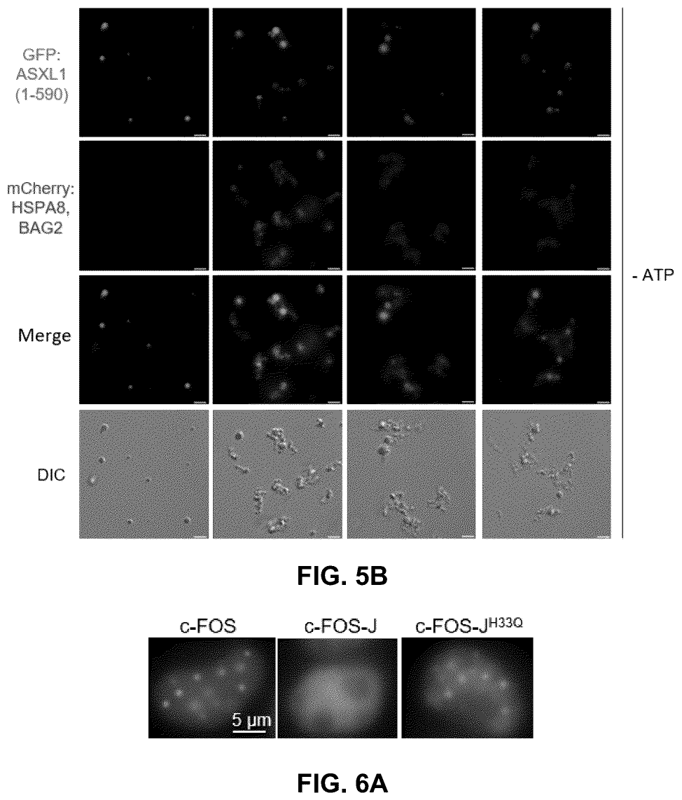

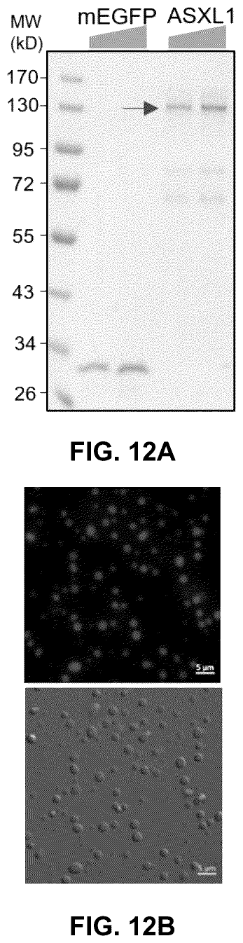

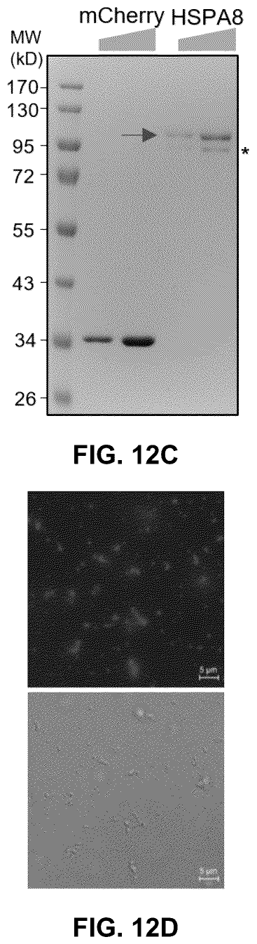

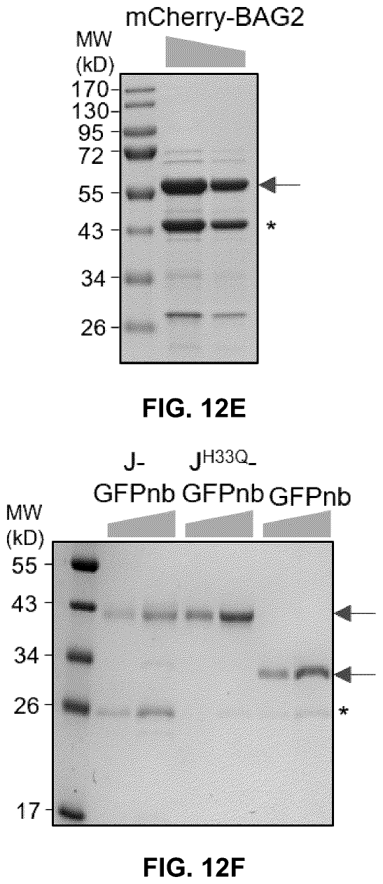

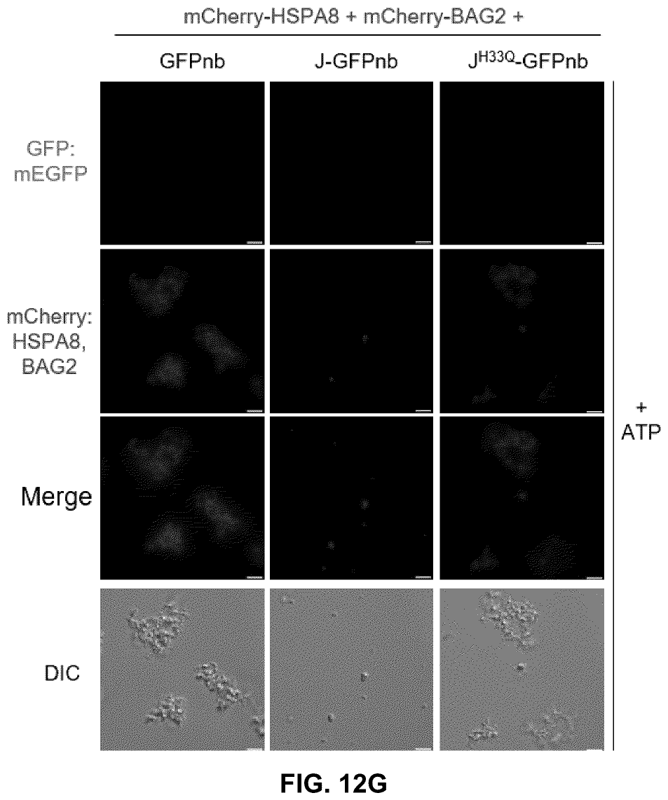

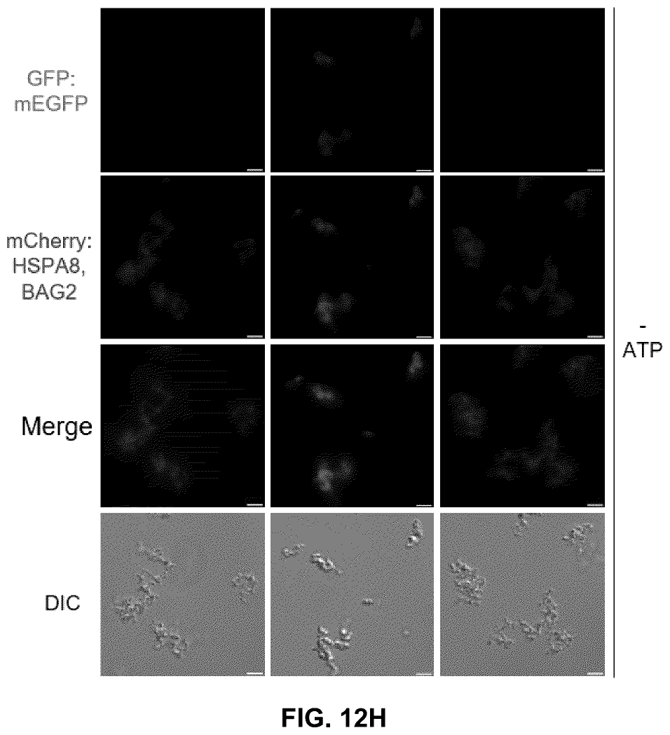

To determine if the chaperoning activity of Hsp70s plays a direct role in dispersing the specific condensates when recruited by the J-nb, dispersal of specific protein condensates by all purified proteins was examined in a fully reconstituted in vitro system (FIGS. 5A-5B and 12A-12H). ASXL1 (1-590), which is the most frequent nonsense mutation of ASXL1 in myeloid malignancies and forms condensates both in vitro and in cells was chosen. ASXL1 (1-590)-mEGFP, mCherry-tagged HSPA8 as the Hsp70, mCherry-tagged BAG2 as the NEF, as well as GFPnb, J-GFPnb, and J^{H33Q}-GFPnb that were not tagged with a fluorescent protein (FIGS. 12A, 12C, 12E, and 12F) were recombinantly expressed and purified. ASXL1 (1-590) indeed formed liquid-like droplets (FIG. 12B), and HSPA8 also formed less liquid-like granules/assemblies (FIG. 12D). ASXL1 (1-590) was first incubated in conditions that allowed its condensate formation. Further incubation with the mCherry control protein retained droplets-like condensates (FIGS. 5A-5B). Further incubation with the dispersal machinery composed of HSPA8, BAG2, and J-GFPnb, in the presence of ATP, greatly reduced ASXL1 condensates (FIG. 5A). In contrast, incubation with either GFPnb or J^{H33Q}-GFPnb (instead of J-GFPnb) in this dispersal machinery did not abolish the ASXL1 condensates, but rather exhibited aggregates that contained ASXL1 and HSP8/BAG2 (but ASXL1 and HSP8/BAG2 did not fully overlap) (FIG. 5A). Such aggregation was likely contributed from HSPA8 and/or ASXL1 (FIGS. 12B and 12D). In the absence of ATP, ASXL1 (1-590) remained in droplet-like morphologies when co-incubated with the dispersal machinery containing either GFPnb, J-GFPnb, or J^{H33Q}-GFPnb (FIG. 5B). These results indicate that J-GFPnb, but not J^{H33Q}-GFPnb, effectively mediates the dispersal of the condensates of a GFP-tagged target protein directly through the chaperoning activity of the Hsp70 in an ATP-dependent manner (which is required by the Hsp70 activity).

Using purified mEGFP protein instead of a specific ASXL1 (1-590)-mEGFP target protein, it was found that the aggregates of HSP8/BAG2 themselves were also largely disassembled by J-GFPnb, but not GFPnb or J^{H33Q}-GFPnb, in a ATP-dependent manner (FIGS. 12G-12H), indicating an intrinsic disassembling ability of the activated dispersal machinery regardless of the identity of the target protein. These results further show the mechanistic basis of the present strategy, and support a broad applicability of this strategy on different target proteins.

### Nanobody-Mediated Targeting of Naturally Occurring Condensates Disrupt Biological Functions

While these data have established the efficacy of dispersing condensates of a tagged protein by using a nb against the tag, this would be informative about the unmodified endogenous target proteins and also would not be viable as a therapeutic strategy. It was next sought to target naturally occurring (un-engineered or modified) endogenous protein condensates by using nbs against the protein itself, starting with a protein that (1) is broadly important in human physiology and disease, (2) forms condensates when expressed from its natural, unmodified genomic locus, and (3) has specific nbs available from public resources.

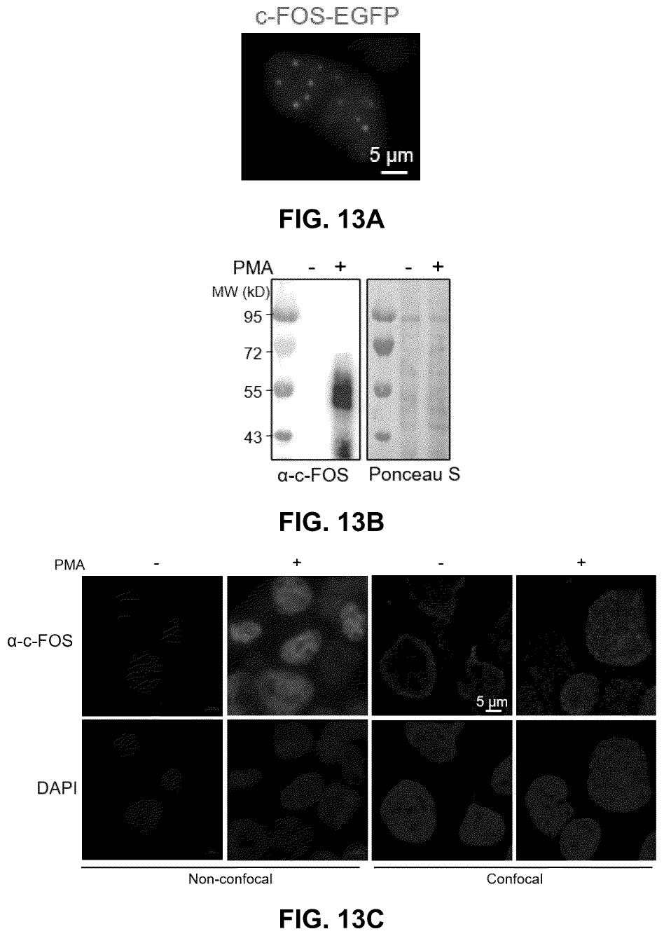

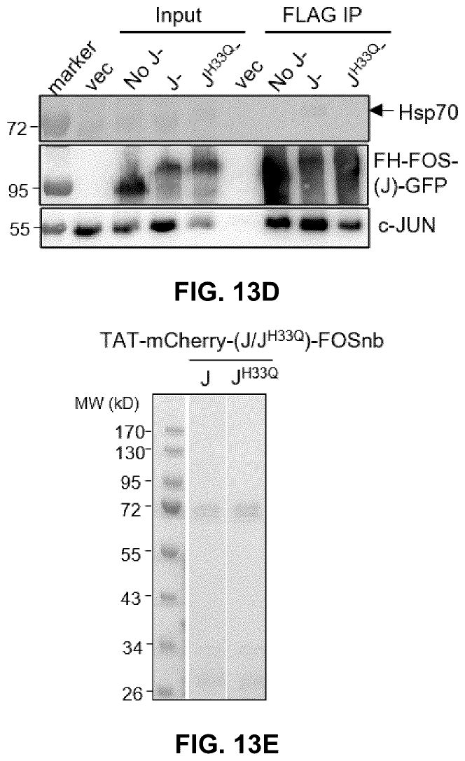

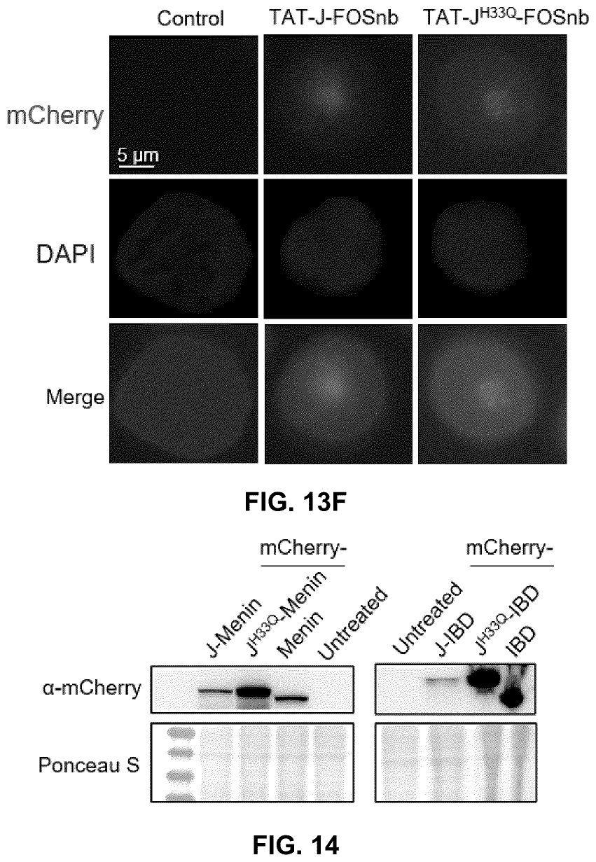

c-FOS is a member of the AP-1 family of transcription factors that regulate numerous biological processes including cell proliferation, differentiation, apoptosis, and transformation. However, it is also challenging to target it. Upon induction by multiple stimuli such as serum, growth factors, phorbol esters and oncogenes, c-FOS forms a heterodimer with c-JUN (88, 89) that bind to TPA-responsive elements or cAMP-response elements to turn on target gene transcription. It has not been reported whether c-FOS forms condensates to regulate transcription. A classical model for the biological activity of c-FOS is the megakaryocytic differentiation of K562 cells, a human proerythroblastic leukemia cell line. Upon protein kinase C activation by phorbol 12-myristate 13-acetate (PMA), K562 cells differentiate into megakaryocytic cells with distinctive morphological features including most prominently the enlarged cell body and extensive vacuolation. AP-1 including c-FOS is upregulated and plays a crucial role in this differentiation process. This data indicated that both transfected and PMA-induced endogenous c-FOS forms nuclear condensates (FIGS. 13A-13C).

It was then shown that c-FOS fused with the J-domain, but not the J^{H33Q} mutant, lost the condensation ability in the nucleus (FIG. 6A), indicating that the c-FOS condensates can also be regulated recruited chaperones. Fusion to the J-domain did not affect its interaction with c-JUN (FIG. 13D). Next, fusion of J-domain or its H33Q mutant was constructed with a nb for c-FOS (FOSnb) from public resources. It was found J-but not J^{H33Q}-fusion with FOSnb substantially dispersed nuclear condensates of these proteins in co-transfected cells (FIG. 6B). Moreover, the c-FOS nuclear puncta of the PMA-induced endogenous c-FOS in K562 cells were dispersed by co-expression of J-FOSnb, but not J^{H33Q}-FOSnb (FIG. 6C). Importantly, PMA-induced megakaryocytic differentiation, as determined by the enlarged cell body and extensive vacuolation, was greatly impaired by J-FOSnb, but not J^{H33Q}-FOSnb, FOSnb itself, or fusion of the J-domain or J^{H33Q} with a non-targeting nb (ALFAnb) (FIGS. 6D-6E).

Purified Cell-Penetrating J-nb Protein Disrupts Biological Activity of Endogenous c-FOS

To explore different methods to efficiently deliver the agents into cells, the effects of a cell-penetrating form of the J-FOSnb protein were tested. J-FOSnb and J^{H33Q}-FOSnb proteins fused with the HIV TAT protein-transduction motif were recombinantly expressed and purified (FIG. 13E), which mediates cell entry of the fused protein. It was found that, when added to cell culture medium, both proteins entered the nucleus of the K562 cells after co-incubation of the proteins with the cells (FIG. 13F). J-FOSnb, but not J^{H33Q}-FOSnb, protein significantly blocked the PMA-induced megakaryocytic differentiation (FIG. 6F), further supporting that dispersing the endogenous c-FOS protein condensates through the molecular chaperones disrupted the function of c-FOS in mediating megakaryocytic differentiation.

Together, these data show the feasibility and biological impact of dispersing natural, un-modified endogenous protein condensates by targeting them to Hsp70s (FIG. 6G).

### A More General Approach to Disperse Unmodified, Endogenous Condensates

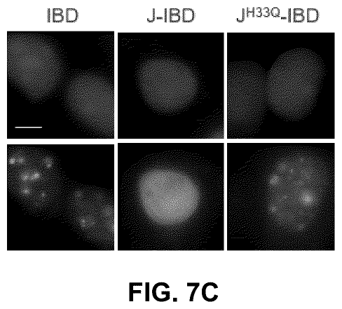

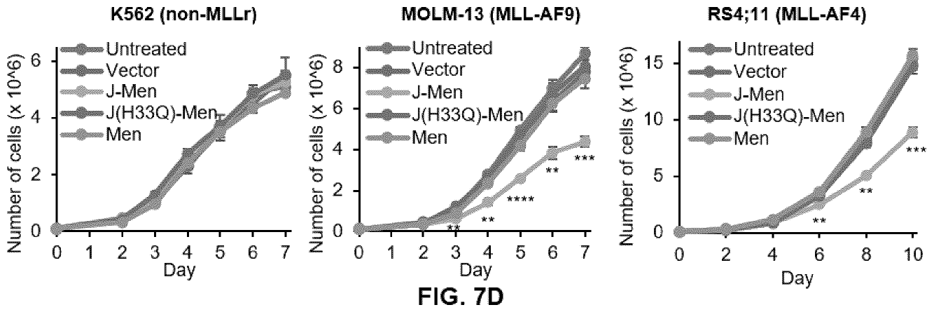

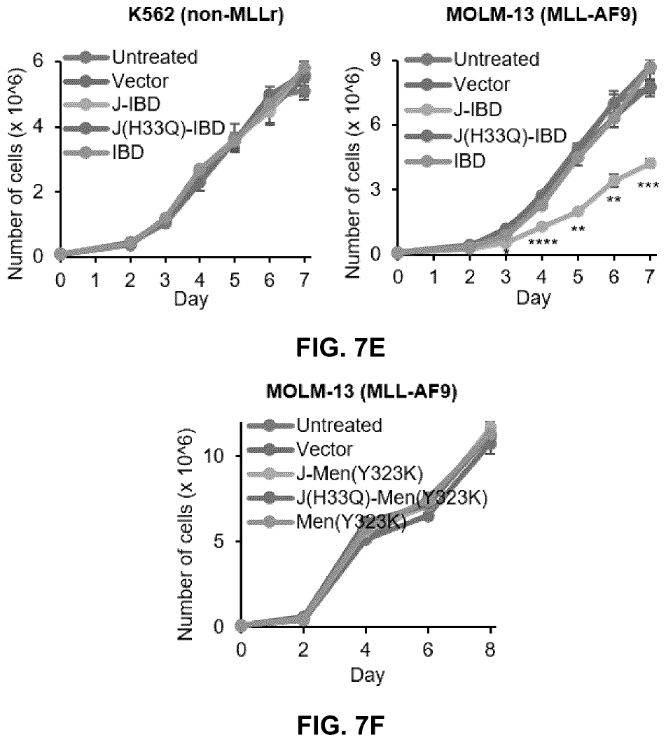

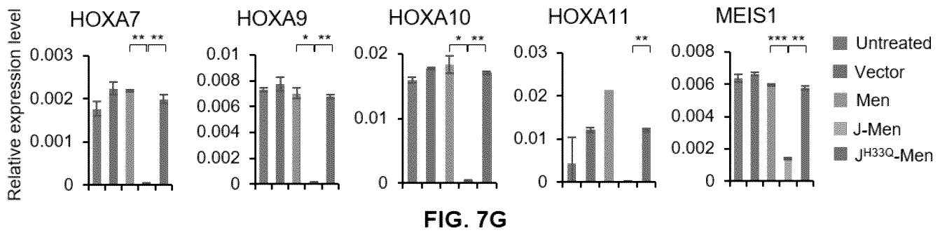

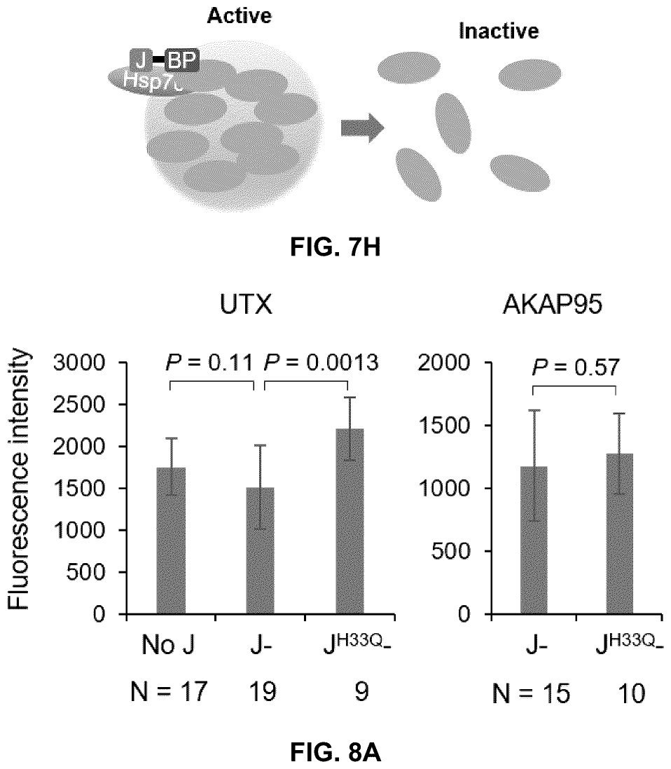

At this moment, most intracellular proteins do not have specific nbs readily available. To make the present strategy more widely feasible for those proteins, it was reasoned that they can be targeted to chaperones by fusing the J-domain to a protein or protein domain that binds to the target protein. This strategy was tested on MLL-AF9. The MLL part of MLL-AF9 has a few established binding proteins including Menin and LEDGF/p75. The near full-length Menin forms a deep pocket that binds a short sequence of MLL, whereas the integrase binding domain (IBD) of LEDGF interact with the MLL-Menin complex. J- orJ^{H33Q}-fusion was thus constructed with either Menin or LEDGF_{IBD}, and determined their effects on MLL-fusion condensates and their ability in driving leukemia. It was found that expression of Menin or LEDGF_{IBD} fused with the J-domain dispersed the condensates of MLL-AF9, while Menin and LEDGF_{IBD} not fused with a J domain and those fused with J^{H33Q} did not affect the condensates (FIGS. 7A and 7C). A single amino acid substitution, Y323K, on Menin introduces a positive charge in the central cavity of Menin and greatly reduced MLL binding by over 100 fold due to electrostatic repulsion, but does not affect the thermodynamic stability of Menin. It was found that the Menin Y323K mutant (Menin^{Y323K}) fused to J domain failed to affect the condensates of the co-expressed MLL-AF9 (FIG. 7B).

As MLL-AF4 and MLL-AF9 are the most prevalent MLL translocations in MLL-driven leukemias, the effects of dispersing the MLL fusion condensates on growth of MOLM-13 and RS4 were tested; 11 leukemia cells driven by MLL-AF9 and MLL-AF4, respectively. While Menin or LEDGF_{IBD}, or their fusions with J^{H33Q}, did not affect the growth of MLL-rearranged leukemia cells, their fusion with the J-domain significantly inhibited the growth of these cells (FIGS. 7D-7E), even though their expression levels were lower than the unfused proteins and J^{H33Q}-fusions in these cells (FIG. 14). These constructs had no effects on the growth K562 cells (FIGS. 7D-7E), which are not driven by MLL rearrangements. It was found that Menin^{Y323K} fused to J domain completely lost the effect on the growth of MOLM-13 cells (FIG. 7F), indicating that binding to MLL is required for a J-domain fusion to affect the growth of the MLL-rearranged leukemia cells. Importantly, J-Menin profoundly reduced the expression of the well-established MLL-AF9 target genes, including HOXA7, HOXA9, HOXA10, HOXA11, and MEIS1, while Menin or J^{H33Q}-Menin did not have any effects on their expression (FIG. 7G). These results suggest that J-Menin inhibits MLL-AF9-driven leukemia growth through dispersing MLL-AF9 condensates and specifically disrupting the function of MLL-AF9 in transcribing oncogenic transcriptional programs.

Taken together, these results suggest that J-domain fused to a target-binding protein or domain can disperse the condensates of the target protein, and disrupt the biological or pathological activity associated with the condensates (FIG. 7H).

### Example 3: Discussion

This study establishes a new strategy to disperse condensates formed by specific, native, endogenous proteins. These data indicate that targeting many different condensate-forming proteins to Hsp70s through J-domain not only disperses the condensates but also disrupts the condensate-dependent biological function, and thus is likely broadly applicable to other protein condensates. The significance of this study is two-fold: (1) this is a loss-of-function approach much needed to understand the functional roles and mechanisms of unmodified, endogenous condensates in a specific biological process in live cells; (2) it lays a foundation for further development of therapeutic strategies by dispersing disease-driving condensates through the cell-intrinsic protein quality surveillance system.

The basic approach currently used to determine the functional importance of condensates is through mutating the condensation-mediating amino acid residues. This is too often by overexpression, which is known to be artifact-prone. Mutating endogenous genes (commonly by CRISPR/Cas9) typically needs to obtain single cell-derived clones, and is thus difficult on primary cells or if the mutations affect cell viability. Moreover, this method is sometimes infeasible or unable to connect to the function of condensation for reasons that follow. (1) Many proteins have extensive IDRs where many different regions may possess condensation property and all contribute to condensation of the whole protein (see further discussion for MLL fusions below). (2) Multi-residue mutations may affect other activities, especially for co-condensation that requires interactions with other proteins for both condensation and activity. This is a particularly challenging issue for especially for condensation that requires heterotypic interactions with other proteins/molecules to drive both condensation and activity (for example, transcription factors with Mediator). Replacement of the region with condensation-mediating regions from unrelated proteins can help, but some replacements do not work due to numerous reasons. The present strategy circumvents these issues.

Engineered systems to induce condensates in cells usually involve fusing the target protein with a protein that can be induced to self-or hetero-associate by light or chemicals. They allow spatiotemporal induction and control of condensates, and when coupled with functional assays, are valuable in causally linking condensation to function. However, they do not address the physiological role of natural condensates in cells, and the induced condensates may not faithfully recapitulate the physicochemical properties of the physiological condensates. In a sense, these methods are analogous to the overexpression/ectopic expression approach in studying gene function, while a better approach would be equivalent to the knockdown/knockout assays. A few methods have recently been shown to dissolve condensates by inducing binding with a ligand or a solubilizing-protein. However, these methods cannot be applied for studying the endogenous and naturally occurring proteins as they are require genetically fusing the target proteins with a protein tag, and are thus also not suitable for potential therapeutic purposes.

Other methods include designing or screening to identify molecules or compounds that can perturb, disperse, or modify the target protein condensates or alter their properties. These studies mainly aim at potential disease treatment, but can also be used for studying condensate function. The identified compounds may affect condensates through unknown mechanisms or get selected based on specific physicochemical properties, which are most likely shared by other proteins in cells. Therefore, their specificity cannot be rigorously demonstrated.

Our strategy in this study is a loss-of-function approach for endogenous and naturally occurring condensates without the need to modify or engineer the target proteins, and thus should greatly help functional studies of the physiological condensates. As a completely different and orthogonal approach, this can be used in combination with the other aforementioned approaches including mutations of the key condensation regions, but should be particularly valuable in cases where other methods are difficult or not feasible, as discussed below.

While many transcription regulators including transcription factors and coactivators are now thought to form transcriptional condensates mediated by their disordered domains (transactivation domains of transcription factors), whether the condensation properties play a functional role in the related biological pathways is a highly debated question. A crucial barrier to a clearer understanding is the inability to separate the amino acid sequences involved in mediating condensate formation from those in binding with other transcription regulators.

While MLL fusion proteins have been documented to be localized to subnuclear compartments, it remains unclear if these fusions have intrinsic ability to form condensates and if their condensation properties are functionally important for their potent leukemogenicity (and would thus provide a rationale for targeting these condensates for therapeutics). It was initially sought to address these questions by the conventional mutational approach as discussed above. In preliminary studies, individual fragments of MLL_{f} (300 amino acids each) were recombinantly expressed, and it was found that many of them had some levels of condensation in vitro (data not shown), suggesting that all these different disordered regions may contribute to the formation of MLL_{f} and MLL-fusions. This makes it very difficult to identify key regions and residues important for condensation and thus difficult to use mutagenesis method to link condensation property to function. That this might be a common challenge to study condensation of other nuclear proteins that have very large IDRs. The profound effects of the J-nb and J-Menin as well as J-LEDGF_{IBD} on cancer cell growth in culture and animal models and on expression of the MLL-fusion target genes strongly support a crucial role of MLL-fusion condensates in driving cancer through regulating oncogenic transcriptional programs. As these effects have been demonstrated on unmodified cancer cells, this method can be further developed into a therapeutically feasible new strategy for combating MLL-rearranged leukemias. Although the abolished growth of the J-ALFAnb-expressing BM precluded efforts in getting sufficient number of cells for chromatin immunoprecipitation assays, it is believed that binding of MLL-fusion with the genomic targets may be impaired upon dispersal of the MLL-fusion condensates by molecular chaperones.

Consistent with a previous report with anti-c-FOS immunostaining signals showing multiple distinct nuclear puncta in cells treated with 12-O-tetradecanoylphorbol-13-acetate (TPA, which induced c-FOS expression), here it is shown that the induced endogenous c-FOS forms nuclear condensates, which are effectively dispersed by J-FOSnb. Importantly, J-FOSnb abolished the biological process that c-FOS is known to mediate as a transcription factor. These results suggest an important biological role of c-FOS transcriptional condensates and set the stage for further mechanistic dissection of the transcriptional changes upon dispersal of the c-FOS condensates.

A key strategy that allows the present method to target the natural occurring protein is the use of either a target-specific nb or a target-binding protein or domain. For targets that do not have a specific nb available or known binding protein/domain, it is expected that the recent boom of computational programs, including AlphaProteo, for de novo design of proteins/domains that bind to a given protein with high affinity and specificity, should greatly help to target specific protein condensates, now that the principle has been demonstrated.

Our strategy takes advantage of the cell-intrinsic protein quality surveillance system, and uses the well evolved J-domain to guide the system to disperse condensates of interest. The direct effect of the condensate dispersal machinery, composed of Hsp70, NEF, and the J-domain-fusion, on the specific target condensates is demonstrated by the in vitro reconstituted system with all purified proteins. The negative control of the point mutations on either the Hsp70-binding (on the J-domain) or the substrate-binding parts (nb or protein) further support the involvement of the chaperones and target specificity. Both the in vitro and in vivo data strong support this model of dispersal of specific condensates by molecular chaperones.

The possibility that the J-domain-recruited Hsp70s perturb other activities of the target proteins in addition to dispersing the condensates cannot be excluded. However, the co-IP assays show that, for all of the target proteins examined here, fusion of the J-domain preserved the binding of the target proteins with their well-established binding partners, though it is unknown if the fused J-domain affects the binding of other proteins. Since orthogonal approaches have shown the functional importance of forming condensates for several proteins in this study (AKAP95, UTX, ASXL1), it is believed that the recruited Hsp70s through the J-domain disrupt the biological function of the target proteins at least in part through dispersing the condensates formed by the proteins. It is noted that no single approach is sufficient to rigorously demonstrate the function of the condensates in a biological process. Rather, a combination of orthogonal approaches should be combined to enhance the confidence of the conclusion that formation of condensates is important for a biological process. Somewhat different from mechanistic studies, therapeutic approaches based on the present strategy may be relatively less concerned on the potential involvement of other mechanisms in addition to condensate dispersal that could together help treat the associated disease.

Our strategy may be considered as a condensate “knockdown” approach much needed today. Given the ubiquitous presence of biomolecular condensates in cellular processes, this approach should find application in a very wide range of research fields. Considering the roles that condensates play in many diseases including cancer, neurodegenerative disorders, infection, and inflammation, it is not hard to envisage that this approach may be potentially developed to treat condensate-driven diseases. Indeed, a particular strengthen of this approach is that it does not need to modify or engineer the endogenous target proteins, making it viable for potential therapeutics.

### Example 4: Methods

### Cells

239T, MDA-MB-231, COS-7 cells were cultured in DMEM (ThermoFisher Scientific) supplemented with 10% fetal bovine serum (FBS, Thermo Fisher Scientific). K562, MOLM-13, and RS4; 11 cells were all cultured in RPMI1640 (ThermoFisher Scientific) supplemented with 10% FBS.

### Constructs

The DNAJB1 and DNAJC7 J-domain cDNAs were cloned from HeLa cell cDNA by PCR and the H33Q mutant was generated by site-directed mutagenesis. The J-domain and mutant fusions were cloned into pcDNA5/FRT/TO (pcDNA5, ThermoFisher Scientific). SV40 nuclear localization signal (NLS) was fused to all constructs to ensure nuclear localization. Some constructs have EGFP (Enhanced Green Fluorescent Protein), mEGFP (monomeric EGFP, with A206K mutation of EGFP), or mCherry fused as indicated. The J-nb plasmids were in the all-in-one doxycycline inducible lentiviral vector pCW57, a gift from Adam Karpf (Addgene plasmid #71782; RRID:Addgene_71782). The GFPnb, a tandem fusion of two anti-GFP nanobodies (GFPenhancer-GGGGS4-LaG16), was a gift from Motoyuki Hattori (Addgene plasmid #140442; RRID:Addgene_140442). The ALFAnb cDNA was synthesized (Biomatik USA, LLC, Delaware, USA) based on the published protein sequence. FOSnb (S1-71) was a gift from James Trimmer (Addgene plasmid #145832; RRID:Addgene_145832). The MSCV-MLL-AF9-EGFP and MSCV-MLL-AF9-ALFA-EGFP plasmids were constructed from the MSCV-MLL-AF9-IRES-EGFP plasmid (a gift from Golam Mohi) through overlapping PCR to remove the stop codon after the AF9 gene and the IRES sequence and, for the ALFA-tagged version, insert the ALFA tag sequence. For protein expression in bacteria, cDNAs were cloned into pET28a with N- or C-terminal tags of 6xHis and mEGFP or mCherry as indicated. The sequences of all plasmids were confirmed by Sanger sequencing.

### Analyses for Protein Disorder

Disordered regions were identified using IUPred and IUPred3.

### Co-Immunoprecipitation Assays

293T cells were transfected with indicated constructs. Twenty-four hours later, cells were lysed in BC300 (50 mM Tris-HCl pH 7.4, 300 mM KCl, 20% glycerol, 0.2 mM EDTA) with 0.1% NP40, 1 mM DTT, and protease inhibitor cocktail (Roche, Cat #4693159001). Lysates were incubated with the anti-FLAG M2 antibody (Sigma, A2220) and washed by the lysis buffer. Bead-bound proteins were resolved by SDS-PAGE and detected by immunoblotting.

### Protein Expression and Purification

The pET28-based constructs were transformed into BL21 Star (DE3) (ThermoFisher Scientific, Cat #C601003) E. coli. Bacteria culture at OD600 of 0.6 were induced with 0.4 mM of Isopropyl β-D-1-thiogalactopyranoside (IPTG) for 5 hours at 25° C. For purifications of 6xHis-tagged proteins, bacterial pellets from 500 mL IPTG-induced culture were resuspended in 6 mL of Lysis Buffer [50 mM Tris-HCl pH 7.3, 450 mM NaCl, 10 mM imidazole, 1× protease inhibitors (Roche, Cat #11836170001)] and lysed by sonication. After centrifugation, the supernatant was incubated with 1 mL of pre-equilibrated Ni-NTA Agarose (Qiagen, Cat #30210) for 1-2 hr at 4° C., washed with the Wash Buffer (50 mM Tris-HCl pH 7.3, 450 mM NaCl, 20 mM imidazole), and eluted with 0.5 mL of Elution Buffer (50 mM Tris-HCl pH 7.3, 450 mM NaCl, 250 mM imidazole) 5 times. Eluates containing proteins were combined and concentrated in Buffer 450 (50 mM Tris-HCl pH 7.3, 450 mM NaCl, 10% Glycerol, 1 mM DTT) using Amicon Ultra-4 centrifugal filter units (Millipore, 30K MWCO, Cat #UFC803008; 50K MWCO, Cat #UFC805008) following manufacturer's instructions. Purified proteins were examined by SDS-PAGE followed by coomassie blue staining. Protein concentration was determined by Nanodrop measurement for OD₂₈₀ and calculation using extinction coefficient provided by ExPASy ProtParam, and also validated by comparing with coomassie blue staining of known concentration of BSA as well. Sequences of protein constructs are provided in Table 1 below.

TABLE 1
Protein Constructs and Sequences

| SEQ | | |
|---|---|---|
| ID | | |
| NO. | Sequence^{a,b,c} | Description |
| 1 | MGKDYYQTLGLARGASDEEIKRAYRRQALRYHPDKNKEP | J domain-linker- |
| | GAEEKFKEIAEAYDVLSDPRKREIFDRYGGGSGGSGGSG | anti-ALFAnb |
| | GVDEVQLQESGGGLVQPGGSLRLSCTASGVTISALNAMA | (dissolves |
| | MGWYRQAPGERRVMVAAVSERGNAMYRESVQGRFTVTR | condensates of |
| | DFTNKMVSLQMDNLKPEDTAVYYCHVLEDRVDSFHDYWG | ALFA-tagged |
| | QGTQVTVSS | proteins) |
| 2 | MGKDYYQTLGLARGASDEEIKRAYRRQALRYHPDKNKEP | J domain-linker- |
| | GAEEKFKEIAEAYDVLSDPRKREIFDRYGGGSGGSGGSG | Menin-NLS from |
| | GVDAMGLKAAQKTLFPLRSIDDVVRLFAAELGREEPDLVLL | cMYC (dissolves |
| | SLVLGFVEHFLAVNRVIPTNVPELTFQPSPAPDPPGGLTYF | MLL-fusion protein |
| | PVADLSIIAALYARFTAQIRGAVDLSLYPREGGVSSRELVKK | condensates) |
| | VSDVIWNSLSRSYFKDRAHIQSLFSFITGTKLDSSGVAFAVV | |
| | GACQALGLRDVHLALSEDHAWVVFGPNGEQTAEVTWHGK | |
| | GNEDRRGQTVNAGVAERSWLYLKGSYMRCDRKMEVAFM | |
| | VCAINPSIDLHTDSLELLQLQQKLLWLLYDLGHLERYPMAL | |
| | GNLADLEELEPTPGRPDPLTLYHKGIASAKTYYRDEHIYPY | |
| | MYLAGYHCRNRNVREALQAWADTATVIQDYNYCREDEEIY | |
| | KEFFEVANDVIPNLLKEAASLLEAGEERPGEQSQGTQSQG | |
| | SALQDPECFAHLLRFYDGICKWEEGSPTPVLHVGWATFLV | |
| | QSLGRFEGQVRQKVRIVSREAEAAEAEEPWGEEAREGRR | |
| | RGPRRESKPEEPPPPKKPALDKGLGTGQGAVSGPPRKPP | |
| | GTVAGTARGPEGGSTAQVPAPAASPPPEGPVLTFQSEKM | |
| | KGMKELLVATKINSSAIKLQLTAQSQVQMKKQKVSTPSDYT | |
| | LSFLKRQRKGLPAAKRVKLD | |
| 3 | MGKDYYQTLGLARGASDEEIKRAYRRQALRYHPDKNKEP | J domain-linker- |
| | GAEEKFKEIAEAYDVLSDPRKREIFDRYGGGSGGSGGSG | LEDGF_{(345-429, IBD)}- |
| | GVDETSMDSRLQRIHAEIKNSLKIDNLDVNRCIEALDELASL | NLS from cMYC |
| | QVTMQQAQKHTEMITTLKKIRRFKVSQVIMEKSTMLYNKFK | (dissolves MLL- |
| | NMFLVPAAKRVKLD | fusion protein |
| | | condensates) |
| 4 | MGKDYYQTLGLARGASDEEIKRAYRRQALRYHPDKNKEP | J domain-linker- |
| | GAEEKFKEIAEAYDVLSDPRKREIFDRYGGGSGGSGGSG | GRB2-NES |
| | GTGMEAIAKYDFKATADDELSFKRGDILKVLNEECDQNWY | (dissolves EMLF- |
| | KAELNGKDGFIPKNYIEMKPHPWFFGKIPRAKAEEMLSKQR | ALK protein |
| | HDGAFLIRESESAPGDFSLSVKFGNDVQHFKVLRDGAGKY | condensates that |
| | FLWVVKFNSLNELVDYHRSTSVSRNQQIFLRDIEQVPQQPT | drive some |
| | YVQALFDFDPQEDGELGFRRGDFIHVMDNSDPNWWKGAC | NSCLCs) |
| | HGQTGMFPRNYVTPVNRNVLPPLERLTL | |
| 5 | MGKDYYQTLGLARGASDEEIKRAYRRQALRYHPDKNKEP | J domain-linker- |
| | GAEEKFKEIAEAYDVLSDPRKREIFDRYGGGSGGSGGSG | GRB2_{SH2}-linker- |
| | GTGWFFGKIPRAKAEEMLSKQRHDGAFLIRESESAPGDFS | NES (dissolves |
| | LSVKFGNDVQHFKVLRDGAGKYFLWVVKFNSLNELVDYHR | EMLF-ALK protein |
| | STSVSRNQQIFLRDIESGRGGSGGSLPPLERLTL | condensates that |
| | | drive some |
| | | NSCLCs) |

^{a}Bold text indicates J domain sequence.
^{b}Underlined text indicates a linker sequence.
^{c}Italicized text in SEQ ID NOs. 2 and 3 indicates a nuclear localization signal and in SEQ ID NOs. 4 and 5 indicates a nuclear export signal.

### In Vitro Condensate Formation and Dispersal Assay

The in vitro condensate dispersal assay was adapted from a previous report on disaggregation of Tau fibrils. Briefly, 2 μM mEGFP or ASXL1 (1-590)-mEGFP was first mixed into the final buffer (50 mM HEPES-KOH, pH 7.5, 50 mM KCl, 25 mM NaCl, 2 mM DTT, 10% PEG6000) to allow ASXL1 condensate formation (due to the lowered salt concentration compared to the protein stock and the presence of PEG6000), and then incubated with either 6.2 μM of mCherry or the dispersal machinery that included mCherry-HSPA8 as the Hsp70 (4 μM), mCherry-BAG2 as the NEF (0.2 μM), and either GFPnb, J-GFPnb, or J^{H33Q}-GFPnb (2 μM each), in a final volume of 10 μL (with 50 mM KCl and 90 mM NaCl due to NaCl from the added protein stocks). The +ATP condition indicates the presence of the energy regeneration system (R&D Systems, Cat #B-10) (final concentrations: 2 mM ATP, 5 mM MgCl₂, 3 U/mL creatine kinase, 10 mM creatine phosphate, 3 U/mL inorganic pyrophosphatase), and the −ATP condition indicates the absence of the energy regeneration system (replaced by the same volume of 5 mM MgCl₂). Reactions were incubated at 30° C. for 1 hr and analyzed by microscopic imaging. For imaging analysis of the in vitro condensates, the reaction was added into a homemade flow chamber comprised of a glass slide sandwiched by a coverslip with one layer of double-sided tape as a spacer. Images were taken on Zeiss Axio Observer Z1 microscope with 63× oil lens. Fluorescent and Differential interference contrast (DIC) microscopy images were taken.

### Antibodies and Chemicals

Antibodies for the following proteins were used: ASXL1 N-terminal (Abcam, ab228009, No RRID but used in literature, and its staining pattern completely overlaps with GFP signal in the FmEGFP-ASXL1^{Y591X} cells in the accompanying manuscript), H3K27me3 (Millipore, 07-449, rabbit), H2AK119ub (Cell Signaling, #8240, RRID:AB_10891618), c-FOS (Cell Signaling, #31254S, rabbit), AKAP95 (Bethyl Laboratories, #A301-062A, rabbit), Hsp70 (Santa Cruz, sc-24, mouse), RbBP5 (Bethyl Laboratories, #A300-109A, rabbit), DDX5 (Bethyl Laboratories, #A300-522A, rabbit), Menin (Bethyl Laboratories, #A300-105A, rabbit), c-JUN (Santa Cruz Biotechnologies, sc-74543, mouse monoclonal), GFP (Abcam, AB13970, chicken), mCherry (Cell Signaling, #43590S, rabbit monoclonal), GAPDH (Millipore #MAB374, RRID:AB_2107445, mouse, 1:5000 for immunoblotting), FLAG (Sigma #A2220, RRID: AB_10063035), HA (Cell signaling, #3724, RRID: AB_1549585, Rabbit, 1:1000 for immunoblotting). AlexaFluor 555 conjugated goat anti-rabbit IgG (Thermo Fisher, A-21428, RRID:AB_141784), and Alexa Fluor 488 conjugated goat anti-mouse IgG (Thermo Fisher, A-11001, RRID:AB_2534069) were also used.

### Viruses

The lentiviral plasmids together with psPAX2 and pMD2.G envelope plasmids at a ratio 4:3:1 were transfected into 293T cells using Lipofectamine 3000 (Thermo Fisher). Six to eight hours later, cells were washed once with PBS and fresh growth medium was added. The viral supernatant from 36-hour post-transfection was concentrated by Lenti-X Concentrator (Takara), then aliquot and stored at −80° C. Cells were transduced with concentrated lentivirus through spin-inoculation at 25° C. at 2200 rpm for 2 hrs each for 3 times with 24 hours interval, and were cultured with antibiotics for selection.

Retroviruses were made by transfection of Platinum-E retroviral packaging cell line (Cell Biolabs, Inc., RV-101) with the retroviral constructs. Two days after transfection, the virus-containing supernatant was collected and virus was concentrated using Retro-X Concentrator (Takara). After 16 hrs incubation at 4° C., retrovirus was pelleted by centrifuging the mixture at 1,500× g for 45 min at 4° C. The retrovirus pellet was gently resuspended in RPMI1640 plus 10% FBS, and immediately used for transduction.

### Reporter Splicing Assays

The double reporter splicing assay was performed as described previously. Briefly, HEK293 cells were transfected with the pTN24 reporter plasmid and indicated plasmids or siRNA using Lipofectamine 2000 reagent (Thermo Fisher Scientific) and collected 48 h after transfection. β-galactosidase and luciferase activities were measured using a Dual-Light System (Applied Biosystems).

### K562 Differentiation Assays

K562 cells were transduced by plnducer20-based lentiviruses followed with G418 selection (500 μg/mL). Stably transduced cells were induced by doxycycline (100 ng/mL) for 24 h and then stimulated by phorbol 12-myristate 13-acetate (PMA) (100 nM) for another 24 h. 0.5×10⁵ cells were collected and deposited onto the glass slide by cytospin (1000 rpm, 5 min), and then subjected to HEMA 3 staining (Fisher Scientific, FIS-23123869). The megakaryocytic differentiation condition was visualized under a brightfield microscope and analyzed using Image J.

The cell-penetrating sequence derived from the HIV TAT protein-transduction motif GYGRKKRRQRRR was fused at the N-terminus of the J-FOSnb. Fused proteins were expressed and purified from BL21. K562 cells were treated with 100 nM of purified protein one day before PMA stimulation and then proceeded to megakaryocytic differentiation assay, 10% glycerol in PBS was used as control.

### Cell Growth, Colony Formation, and Transplant Assays

For cell growth assays, cells were seeded in 24-well plate. Cell number was manually counted on indicated days.

All animal procedures were approved by the Institutional Animal Care and Use Committee at the University of Virginia. All mice were maintained under specific pathogen-free conditions and housed in individually ventilated cages.

For BM colony formation assays, BM was harvested from 6-8 weeks old female C57BL/6 mice (Jackson Laboratory, #000664). Lineage-depleted and c-kit-positive cells were selected using Lineage Cell Depletion Kit (Miltenyi Biotec, cat #130-090-858) and CD117 (c-kit) Microbeads (Miltenyi Biotec, cat #130-091-224), and incubated overnight in RPMI 1640 with 10% fetal bovine serum, containing 50 ng/ml SCF, 50 ng/ml Flt-3, and 50 ng/mL TPO (all from Peprotech, Rocky Hill, NJ) at 37° C. to promote cell cycle entry. Cells were then transduced with concentrated retroviruses by incubation for 60 hours. The transduced cells were either sorted by flow cytometry or selected by proper antibiotics, and were used in colony formation and transplant assays. For colony formation, the selected cells were plated into MethoCult (Stemcell Technologies, M3234) supplemented with 10 ng/mL IL-3.

For BM transplant assays, mouse BM cells were isolated from the femurs and tibias of 8-week old C57BL/6 (CD45.2⁺) female mice 4 days after intraperitoneal administration of 150 mg/kg 5-fluorourcil (5-FU), and cultured overnight in RPMI1640 medium supplemented with 10% FBS, 50 ng/ml SCF, 50 ng/mL Flt-3, and 50 ng/ml TPO. The pre-stimulated cells then were transduced for 60 hours with retroviruses. The transduced BM cells were FACS-sorted. GFP and mCherry double positive cells were performed on SONY MA900 Cell Sorter. 100 μm sorting chips were used. All cell sorting steps followed the manufacturer's instructions. Then 1×10⁵ sorted cells were injected through tail vein into 10-week old CD45.1⁺ C57BL/6 recipient mice that had been administered with a sublethal dose of 5.25 Gy total-body x-ray irradiation. Hematology of peripheral blood of transplanted mice was measured using HEMAVET 950FS. Scatter plots were generated in R (v4.3.1).

### Immunofluorescence and Live Cell Imaging

For immunofluorescence, cells were washed with PBS and fixed with ice cold methanol for 15 min. The fixed cells were incubated with 1:250 rabbit antibodies overnight at 4° C., and developed with 1: 300 Alexa Fluor 555 conjugated goat anti-rabbit secondary antibody for 1 hr at room temperature. The slides were further counterstained with DAPI.

Immunofluorescence assays for H2A deubiquitination and H3K27 demethylation were performed as described in the accompanying manuscript and. Cells on 35 mm glass-bottom dishes (MatTek) were transfected using Lipofectamine 3000 (ThermoFisher Scientific, Cat #L3000015). After about 40 hr, cells were washed with PBS, fixed in 4% paraformaldehyde in PBS, permeabilized in 0.5% Triton X-100 in PBS, and blocked in 1% BSA in PBST. The fixed cells were incubated with the primary antibodies overnight at 4° C., and developed with the mixture of Alexa Fluor 555 conjugated goat anti-rabbit secondary antibody (1:1000) (ThermoFisher Scientific, A-21428) and Alexa Fluor 488 conjugated goat anti-mouse secondary antibody (1:1000) (ThermoFisher Scientific, A-11001) for 1 hr at room temperature. Cells were further counterstained with DAPI. Images were acquired on Zeiss fluorescence microscope with 63× oil lens.

For live cell imaging, cells were cultured in 35 mm glass-bottom dishes (MatTek), and used for imaging on Zeiss fluorescence microscope with 63× oil lens or Zeiss LSM780 confocal microscope supported with a Chamlide TC temperature, humidity and CO₂ chamber. Images were collected by either 40× or 60× oil lens.

### RT-qPCR and RNA-Seq Analysis

Total RNA was prepared using Qiagen RNeasy Plus Mini Kit. The first-strand cDNA was prepared using Invitrogen SuperScript III First-Strand Synthesis System for RT-PCR (Invitrogen, 18080051). QPCR was prepared using Takara TB Green Advantage (Takara, #639676) and performed on Bio-Rad CFX384 Real-Time System.

Qualities of total RNA were assessed using Qubit RNA IQ Assay Kits with an Agilent 2100 Bioanalyzer. Samples with RNA IQ scores higher than 9 were further processed to library preparation. mRNA was isolated using NEBNext Poly (A) mRNA Magnetic Isolation Module (New England Biolab, E7490S). Libraries of the resulting mRNA were prepared using NEBNext Ultra II Directional RNA Library Prep Kit for Illumina (New England Biolab, E7760S). Adapters and barcodes were added to the libraries using NEBNext Multiplex Oligos for Illumina (New England Biolab, E7600S). All samples were sequenced on an Illumina NovaSeq X Plus platform and 2×150 bp paired-end reads were collected.

RNA-seq reads were mapped to the USCS mouse reference genome mm39 with GENCODE vM33 using STAR (v2.7.9a). Low quality mapped reads (Q<30) were removed from the analysis. Read count tables were generated using RSEM (Li and Dewey, 2011) (v.1.3.0). Differential expression (DE) analyses were performed using EdgeR (v4.2.2). Heatmaps were generated in R (v.4.3.1). GSEA analysis were performed using GSEA (v4.3.3) (Broad Institute, Cambridge, MA, USA) software package.

### Quantification and Statistical Analysis

Statistical parameters including the definitions and exact values of n (e.g., number of experiments, number of cells, number of colonies, etc), distributions and deviations are reported in the Brief Description of the Drawings. All data were expressed as average ±SD; P values by 2-tailed unpaired Student's t-test as indicated in the Brief Description of the Drawings. Statistical analysis was performed by Excel, or Python. A P value of less than 0.05 was considered significant.

### Example 5: Non-Small Cell Lung Cancer

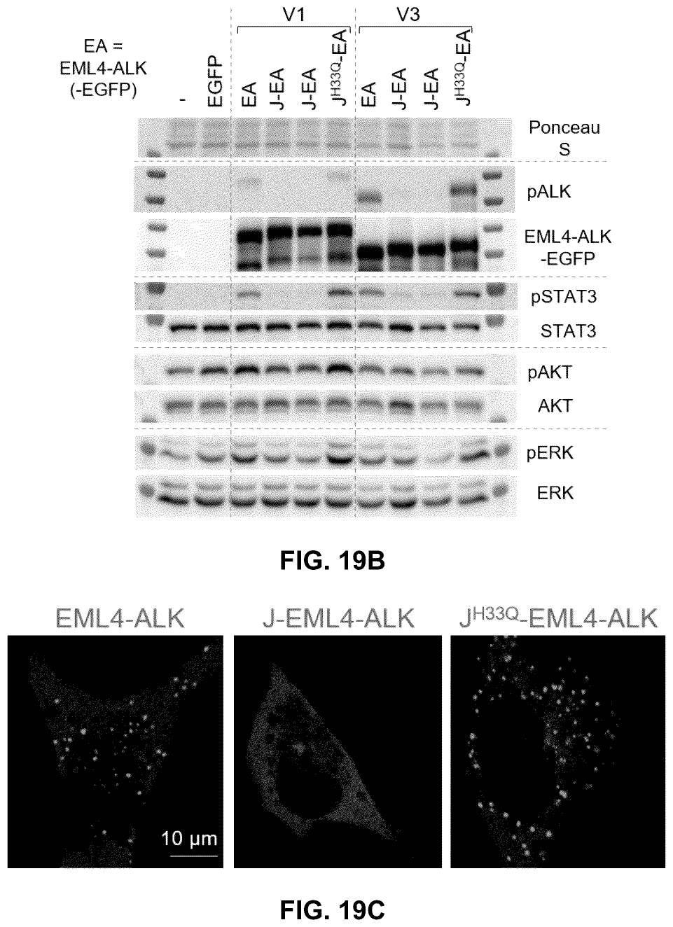

In addition to blood cancers, condensates have been shown to drive other disease processes such as, for example, in non-small cell lung cancer (NSCLC). It was tested whether the same strategy adopted for blood cancers can work for other condensate driven pathologies. FIGS. 19A-19C show that receptor tyrosine kinase (RTK) fusions, including EML4-ALK, are important drivers of non-small cell lung cancer (NSCLC). Although tyrosine kinase inhibitors (TKIs) are used as a first-line treatment for NSCLC, resistant mutations often arise. EML4-ALK forms cytoplasmic condensates to drive cancer growth through constitutively active signaling. FIG. 19A: exemplary RTK fusion. FIG. 19B: testing of J-domain fusions to inhibit condensate formation in various proteins. FIG. 19C: disrupting condensates in Beas2B cells, an immortalized cell line derived from normal human bronchial epithelium.

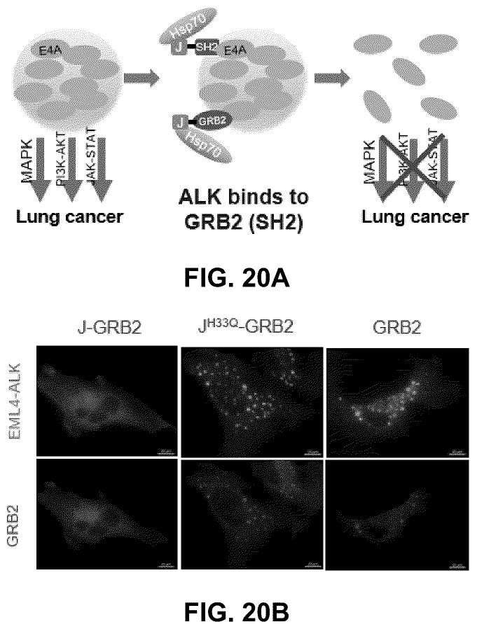

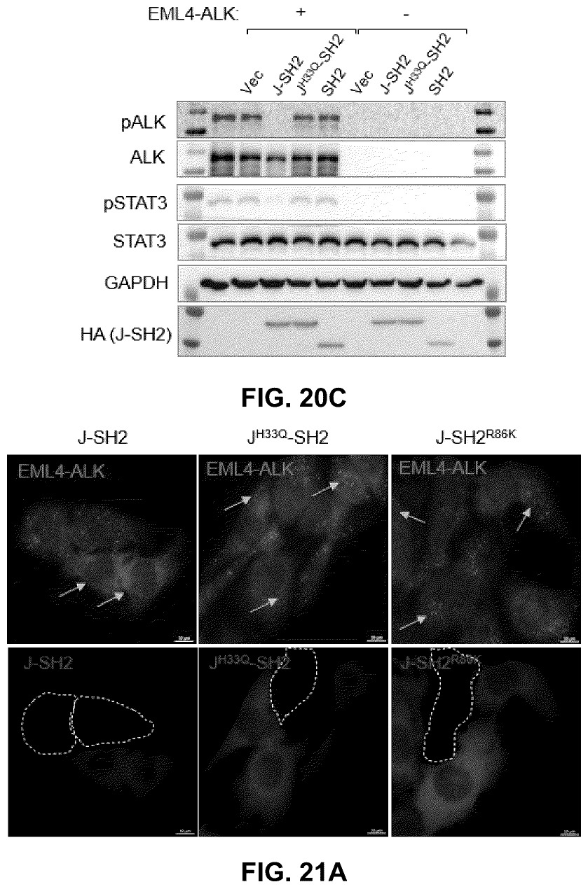

FIGS. 20A-20C show targeting unmodified EMLF-ALK condensates disrupts signaling. FIG. 20A: schematic model of condensate disruption. FIG. 20B: disruption of condensates in Beas2B cells. FIG. 20C: The disclosed strategy successfully targets EML4-ALK condensates.

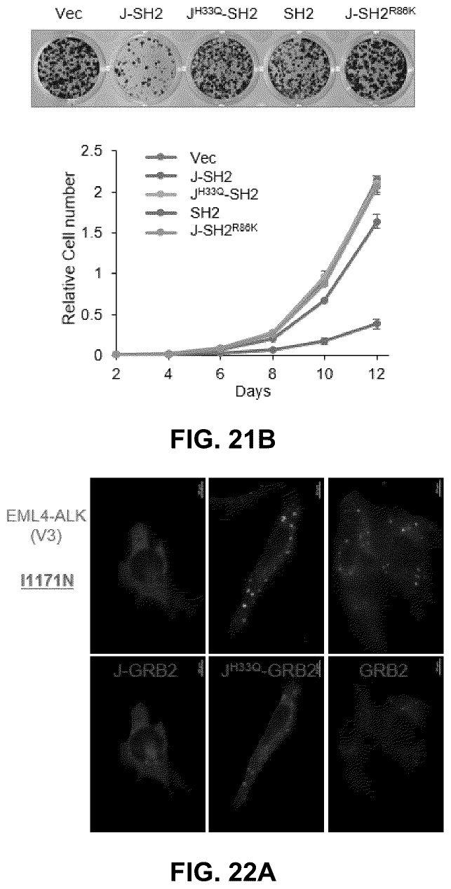

FIGS. 21A-21B show that dissolving endogenous, unmodified EML4-ALK condensates inhibits patient-derived cancer cell growth. FIG. 21A: fluorescence microscopy. FIG. 21B: relative cell number for different J-domain wt and mutant fusion proteins. Cells are NCI-H3122 cells from an EML4-ALK driven NSCLC patient. The R86K mutation disrupts SH2 binding with phosphorylated tyrosine residues.

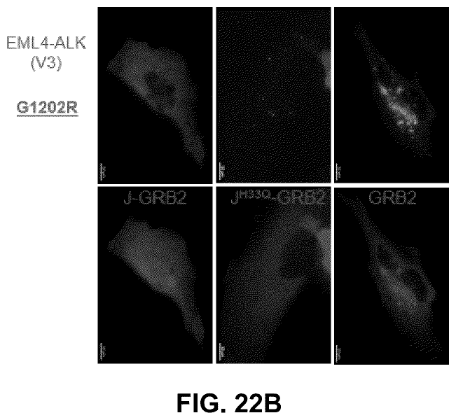

FIGS. 22A-22B show dissolving condensates of TKI-resistant mutants of EML4-ALK. FIG. 22A: Beas2B cells with 11171N mutation. FIG. 22B: Beas2B cells with G1202R mutation.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Abdel-Wahab O, et al. ASXL1 mutations promote myeloid transformation through loss of PRC2-mediated gene repression. Cancer Cell. 2012; 22(2):180-193.
2. Abdel-Wahab O, et al. Deletion of Asxl1 results in myelodysplasia and severe developmental defects in vivo. J Exp Med. 2013; 210(12):2641-2659.
3. Alberti, S., et al. (2019). Liquid-Liquid Phase Separation in Disease. Annual review of genetics 53, 171-194.
4. Alberti, S., et al. (2019). Considerations and Challenges in Studying Liquid-Liquid Phase Separation and Biomolecular Condensates. Cell 176, 419-434.
5. Alberti, S., et al. (2021). Biomolecular condensates at the nexus of cellular stress, protein aggregation disease and ageing. Nature reviews Molecular cell biology 22, 196-213.
6. Asada S, et al. The role of ASXL1 in hematopoiesis and myeloid malignancies. Cell Mol Life Sci. 2019; 76(13):2511-2523.
7. Banani, S.F., et al. (2022). Genetic variation associated with condensate dysregulation in disease. Developmental cell 57, 1776-1788.e1778.
8. Banani, S.F., et al. (2017). Biomolecular condensates: organizers of cellular biochemistry. Nature reviews Molecular cell biology 18, 285-298.
9. Barger, C.J., et al. (2019). Pan-Cancer Analyses Reveal Genomic Features of FOXM1 Overexpression in Cancer. Cancers 11.
10. Bennett, N.R., et al. (2023). Improving de novo protein binder design with deep learning. Nature communications 14, 2625.
11. Boczek, E.E., et al. (2021). HspB8 prevents aberrant phase transitions of FUS by chaperoning its folded RNA-binding domain. eLife 10.
12. Boija, A., et al. (2018). Transcription Factors Activate Genes through the Phase-Separation Capacity of Their Activation Domains. Cell 175, 1842-1855.e1816.
13. Boija, A., et al. (2021). Biomolecular Condensates and Cancer. Cancer cell 39, 174-192.
14. Bracha, D., et al. (2019). Probing and engineering liquid-phase organelles. Nature biotechnology 37, 1435-1445.
15. Brumbaugh-Reed, E.H., et al. (2024). Rapid and reversible dissolution of biomolecular condensates using light-controlled recruitment of a solubility tag. Nature communications 15, 6717.
16. Cao, L., et al. (2022). Design of protein-binding proteins from the target structure alone. Nature 605, 551-560.
17. Caslini, C., et al. (2007). Interaction of MLL amino terminal sequences with menin is required for transformation. Cancer research 67, 7275-7283.
18. Cermáková, K., et al. (2014). Validation and structural characterization of the LEDGF/p75-MLL interface as a new target for the treatment of MLL-dependent leukemia. Cancer Res 74, 5139-5151.
19. Chong, S., et al. (2018). Imaging dynamic and selective low-complexity domain interactions that control gene transcription. Science (New York, NY) 361, aar2555.
20. Chu, Y., et al. (2019). Six1 regulates leukemia stem cell maintenance in acute myeloid leukemia. Cancer science 110, 2200-2210.
21. Conti, B.A., et al. (2022). Biomolecular condensates: new opportunities for drug discovery and RNA therapeutics. Trends in pharmacological sciences 43, 820-837
22. Dobin, A., et al. (2013). STAR: ultrafast universal RNA-seq aligner. Bioinformatics (Oxford, England) 29, 15-21.
23. Dong, J.X., et al. (2019). A toolbox of nanobodies developed and validated for use as intrabodies and nanoscale immunolabels in mammalian brain neurons. eLife 8.
24. Eriksson, M., et al. (2005). AP-1 regulates alpha2beta1 integrin expression by ERK-dependent signals during megakaryocytic differentiation of K562 cells. Experimental cell research 304, 175-186.
25. Faust, O., et al. (2020). Structural and Biochemical Properties of Hsp40/Hsp70 Chaperone System. Advances in experimental medicine and biology 1243, 3-20.
26. Fridy, P.C., et al. (2014). A robust pipeline for rapid production of versatile nanobody repertoires. Nature methods 11, 1253-1260.
27. Gao, X., et al. (2015). Human Hsp70 Disaggregase Reverses Parkinson's-Linked a-Synuclein Amyloid Fibrils. Molecular cell 59, 781-793.
28. Gelsi-Boyer V, et al. Mutations in ASXL1 are associated with poor prognosis across the spectrum of malignant myeloid diseases. J Hematol Oncol. 2012; 5:12.
29. Ghisi, M., et al. (2016). Id2 and E Proteins Orchestrate the Initiation and Maintenance of MLL-Rearranged Acute Myeloid Leukemia. Cancer Cell 30, 59-74.
30. Gibson, B.A., et al. (2019). Organization of Chromatin by Intrinsic and Regulated Phase Separation. Cell 179, 470-484.e421.
31. Götzke, H., et al. (2019). The ALFA-tag is a highly versatile tool for nanobody-based bioscience applications. Nature communications 10, 4403.
32. Gu, J., et al. (2020). Hsp40 proteins phase separate to chaperone the assembly and maintenance of membraneless organelles. Proceedings of the National Academy of Sciences of the United States of America 117, 31123-31133.
33. Gu, J., et al. (2021). Hsp70 chaperones TDP-43 in dynamic, liquid-like phase and prevents it from amyloid aggregation. Cell research 31, 1024-1027.
34. Hartl, F.U., et al. (2011). Molecular chaperones in protein folding and proteostasis. Nature 475, 324-332.
35. Hernandez-Candia, C.N., et al. (2024). A platform to induce and mature biomolecular condensates using chemicals and light. Nature chemical biology 20, 452-462.
36. Hernández-Candia, C.N., et al. (2021). A modular tool to query and inducibly disrupt biomolecular condensates. Nature communications 12, 1809.
37. Hnisz, D., et al. (2017). A Phase Separation Model for Transcriptional Control. Cell 169, 13-23.
38. Hu, J., et al. (2016). AKAP95 regulates splicing through scaffolding RNAs and RNA processing factors. Nature communications 7, 13347.
39. Huang, J., et al. (2012). The same pocket in menin binds both MLL and JUND but has opposite effects on transcription. Nature 482, 542-546.
40. Huether, R., et al. (2014). The landscape of somatic mutations in epigenetic regulators across 1,000 paediatric cancer genomes. Nature communications 5, 3630.
41. Hyman, A.A., et al. (2014). Liquid-liquid phase separation in biology. Annual review of cell and developmental biology 30, 39-58.
42. Igelmann, S., et al. (2022). Liquid-Liquid Phase Separation in Cancer Signaling, Metabolism and Anticancer Therapy. Cancers 14.
43. Jevtic, Z., et al. (2023). Biomolecular Condensates in Myeloid Leukemia: What Do They Tell Us? HemaSphere 7, e923.
44. Kato, M., et al. (2012). Cell-free formation of RNA granules: low complexity sequence domains form dynamic fibers within hydrogels. Cell 149, 753-767.
45. Krivtsov, A.V., et al. (2007). MLL translocations, histone modifications and leukaemia stem-cell development. Nat Rev Cancer 7, 823-833.
46. Kwon, I., et al. (2013). Phosphorylation-regulated binding of RNA polymerase II to fibrous polymers of low-complexity domains. Cell 155, 1049-1060.
47. Larson, A.G., et al. (2017). Liquid droplet formation by HP1alpha suggests a role for phase separation in heterochromatin. Nature 547, 236-240.
48. Li, B., et al. (2011). RSEM: accurate transcript quantification from RNA-Seq data with or without a reference genome. BMC bioinformatics 12, 323.
49. Li, B.E., et al. (2014). Two decades of leukemia oncoprotein epistasis: the MLL1 paradigm for epigenetic deregulation in leukemia. Experimental hematology 42, 995-1012.
50. Li, D., et al. (2022). Spatiotemporal dynamic regulation of membraneless organelles by chaperone networks. Trends in cell biology 32, 1-3.
51. Li, W., et al. (2020). Biophysical properties of AKAP95 protein condensates regulate splicing and tumorigenesis. Nature cell biology 22, 960-972.
52. Li, W., et al. (2021). Nuclear Protein Condensates and Their Properties in Regulation of Gene Expression. Journal of molecular biology, 167151.
53. Liu, Q., et al. (2020a). Structural and functional analysis of the Hsp70/Hsp40 chaperone system. Protein science: a publication of the Protein Society 29, 378-390.
54. Liu, Z., et al. (2020b). Hsp27 chaperones FUS phase separation under the modulation of stress-induced phosphorylation. Nature structural & molecular biology 27, 363-372.
55. Lu, H., et al. (2018). Phase-separation mechanism for C-terminal hyperphosphorylation of RNA polymerase II. Nature 558, 318-323.
56. Lu, S., et al. (2022). Heat-shock chaperone HSPB1 regulates cytoplasmic TDP-43 phase separation and liquid-to-gel transition. Nature cell biology 24, 1378-1393.
57. Lyon, A.S., et al. (2021). A framework for understanding the functions of biomolecular condensates across scales. Nature reviews Molecular cell biology 22, 215-235.
58. Matsuo, Y., et al. (1997). Two acute monocytic leukemia (AML-M5a) cell lines (MOLM-13 and MOLM-14) with interclonal phenotypic heterogeneity showing MLL-AF9 fusion resulting from an occult chromosome insertion, ins (11; 9) (q23; p22p23). Leukemia 11, 1469-1477.
59. McSwiggen, D.T., et al. (2019). Evaluating phase separation in live cells: diagnosis, caveats, and functional consequences. Genes & development 33, 1619-1634.
60. Mehta, S., et al. (2022). Liquid-liquid phase separation drives cellular function and dysfunction in cancer. Nature reviews Cancer 22, 239-252.
61. Mitrea, D.M., et al. (2022). Modulating biomolecular condensates: a novel approach to drug discovery. Nature reviews Drug discovery 21, 841-862.
62. Mogk, A., et al. (2018). Cellular Handling of Protein Aggregates by Disaggregation Machines. Molecular cell 69, 214-226.
63. Mullighan, C.G., et al. (2007). Pediatric acute myeloid leukemia with NPM1 mutations is characterized by a gene expression profile with dysregulated HOX gene expression distinct from MLL-rearranged leukemias. Leukemia 21, 2000-2009.
64. Muntean, A.G., et al. (2012). The pathogenesis of mixed-lineage leukemia. Annual review of pathology 7, 283-301.
65. Murai, M.J., et al. (2011). Crystal structure of menin reveals binding site for mixed lineage leukemia (MLL) protein. J Biol Chem 286, 31742-31748.
66. Murai, M.J., et al. (2014). The same site on the integrase-binding domain of lens epithelium-derived growth factor is a therapeutic target for MLL leukemia and HIV. Blood 124, 3730-3737.
67. Nachman, E., et al. (2020). Disassembly of Tau fibrils by the human Hsp70 disaggregation machinery generates small seeding-competent species. The Journal of biological chemistry 295, 9676-9690.
68. Nakamura, H., et al. (2019). Harnessing biomolecular condensates in living cells. Journal of biochemistry 166, 13-27.
69. Narlikar, G.J., et al. (2021). Is transcriptional regulation just going through a phase? Molecular cell 81, 1579-1585.
70. Olsen, S.N., et al. (2022). MLL::AF9 degradation induces rapid changes in transcriptional elongation and subsequent loss of an active chromatin landscape. Mol Cell 82, 1140-1155.e1111.
71. Panuzzo C, et al. Landscape of Tumor Suppressor Mutations in Acute Myeloid Leukemia. J Clin Med. 2020; 9 (3).
72. Patel, A., et al. (2022). Principles and functions of condensate modifying drugs. Frontiers in molecular biosciences 9, 1007744.
73. Robinson, M.D., et al. (2010). edgeR: a Bioconductor package for differential expression analysis of digital gene expression data. Bioinformatics (Oxford, England) 26, 139-140.
74. Rosenzweig, R., et al. (2019). The Hsp70 chaperone network. Nature reviews Molecular cell biology 20, 665-680.
75. Ross, M.E., et al. (2004). Gene expression profiling of pediatric acute myelogenous leukemia. Blood 104, 3679-3687.
76. Sabari, B.R., et al. (2018). Coactivator condensation at super-enhancers links phase separation and gene control. Science (New York, NY) 361, aar3958.
77. Sasaki, T., et al. (2006). Spatiotemporal regulation of c-Fos by ERK5 and the E3 ubiquitin ligase UBR1, and its biological role. Molecular cell 24, 63-75.
78. Schwarze, S.R., et al. (1999). In vivo protein transduction: delivery of a biologically active protein into the mouse. Science (New York, NY) 285, 1569-1572.
79. Shaulian, E., et al. (2002). AP-1 as a regulator of cell life and death. Nature cell biology 4, E131-136.
80. Shi, B., et al. (2021). UTX condensation underlies its tumour-suppressive activity. Nature 597, 726-731.
81. Shin, Y., et al. (2017). Liquid phase condensation in cell physiology and disease. Science (New York, NY) 357, eaaf4382.
82. Shin, Y., et al. (2018). Liquid Nuclear Condensates Mechanically Sense and Restructure the Genome. Cell 175, 1481-1491.e1413.
83. Snead, W.T., et al. (2019). The Control Centers of Biomolecular Phase Separation: How Membrane Surfaces, PTMs, and Active Processes Regulate Condensation. Molecular cell 76, 295-305.
84. Stong, R.C., et al. (1985). Human acute leukemia cell line with the t(4;11) chromosomal rearrangement exhibits B lineage and monocytic characteristics. Blood 65, 21-31.
85. Stortz, M., et al. (2024). Transcriptional condensates: a blessing or a curse for gene regulation? Communications biology 7, 187.
86. Strom, A.R., et al. (2017). Phase separation drives heterochromatin domain formation. Nature 547, 241-245.
87. Tan, F., et al. (2010). Essential role for ALCAM gene silencing in megakaryocytic differentiation of K562 cells. BMC molecular biology 11, 91.
88. Tripathi, S., et al. (2023). Defining the condensate landscape of fusion oncoproteins. Nature communications 14, 6008.
89. Trojanowski, J., et al. (2022). Transcription activation is enhanced by multivalent interactions independent of phase separation. Molecular cell 82, 1878-1893.e1810.
90. Tulpule, A., et al. (2021). Kinase-mediated RAS signaling via membraneless cytoplasmic protein granules. Cell 184, 2649-2664.e2618.
91. Turnham, R.E., et al. (2019). An acquired scaffolding function of the DNAJ-PKAc fusion contributes to oncogenic signaling in fibrolamellar carcinoma. eLife 8.
92. Vannucchi AM, et al. Epigenetics and mutations in chronic myeloproliferative neoplasms. Haematologica. 2011; 96(10):1398-1402.
93. Vázquez Torres, S., et al. (2024). De novo design of high-affinity binders of bioactive helical peptides. Nature 626, 435-442.
94. Wang J, et al. Loss of Asxl1 leads to myelodysplastic syndrome-like disease in mice. Blood. 2014; 123(4):541-553.
95. Wang, Q.F., et al. (2011). MLL fusion proteins preferentially regulate a subset of wild-type MLL target genes in the leukemic genome. Blood 117, 6895-6905.
96. Watson, J.L., et al. (2023). De novo design of protein structure and function with RFdiffusion. Nature 620, 1089-1100.
97. Woodruff, J.B., et al. (2018). Organization and Function of Non-dynamic Biomolecular Condensates. Trends in biochemical sciences 43, 81-94.
98. Xia, Y.K., et al. (2021). Tumor-derived neomorphic mutations in ASXL1 impairs the BAP1-ASXL1 -FOXK1/K2 transcription network. Protein & cell 12, 557-577.
99. Yamamoto, K., et al. (2021). A histone modifier, ASXL1, interacts with NONO and is involved in paraspeckle formation in hematopoietic cells. Cell reports 36, 109576.
100. Yokoyama, A., et al. (2008). Menin critically links MLL proteins with LEDGF on cancer-associated target genes. Cancer Cell 14, 36-46.
101. Yokoyama, A., et al. (2005). The menin tumor suppressor protein is an essential oncogenic cofactor for MLL-associated leukemogenesis. Cell 123, 207-218.
102. Yoo, H., et al. (2022). Chaperones directly and efficiently disperse stress-triggered biomolecular condensates. Molecular cell 82, 741-755.e711.
103. Yu, H., et al. (2021). HSP70 chaperones RNA-free TDP-43 into anisotropic intranuclear liquid spherical shells. Science (New York, NY) 371.
104. Zambaldi, V., et al. (2024). De novo design of high-affinity protein binders with AlphaProteo, pp. arXiv:2409.08022.
105. Zeng J, Li HC, et al. Selection of human single domain antibodies recognizing the CMYC protein using enhanced intracellular antibody capture. J Immunol Methods. 2015;426:140-143.
106. Zhang, J.Z., et al. (2020a). Phase Separation of a PKA Regulatory Subunit Controls cAMP Compartmentation and Oncogenic Signaling. Cell 182, 1531-1544.e1515.
107. Zhang, Z., et al. (2020b). Structure-based engineering of anti-GFP nanobody tandems as ultra-high-affinity reagents for purification. Scientific reports 10, 6239.
108. Zhu, G., et al. (2020). Phase Separation of Disease-Associated SHP2 Mutants Underlies MAPK Hyperactivation. Cell 183, 490-502.e418.

What is claimed is:

1. A method for treating or preventing a disease associated with aberrant condensation of a biomolecule in a subject, the method comprising contacting one or more cells in the subject with a fusion protein comprising a J domain protein and a targeting molecule, wherein the targeting molecule binds the biomolecule.

2. The method of claim 1, wherein when the targeting molecule binds the biomolecule, the J domain protein recruits a chaperone protein to disassemble the condensate.

3. The method of claim 1, wherein the biomolecule comprises a target protein.

4. The method of claim 3, wherein the target protein comprises a truncated mutant, one or more intrinsically disordered regions, or any combination thereof.

5. The method of claim 3, wherein the target protein comprises MLL-AF9, another MLL-fusion condensate, ASXL1, EML4-ALK, or any combination thereof.

6. The method of claim 1, wherein the chaperone protein comprises HSP70.

7. The method of claim 1, wherein the targeting molecule comprises a nanobody or a target protein binding domain.

8. The method of claim 1, wherein the subject is a mammal.

9. The method of claim 1, wherein the disease comprises a cancer selected from a blood cancer or non-small cell lung cancer (NSCLC).

10. The method of claim 9, wherein the blood cancer comprises leukemia.

11. The method of claim 10, wherein the blood cancer is leukemia and the target protein is a truncated mutant of ASXL1.

12. The method of claim 9, wherein the cancer is NSCLC and the target protein is EML4-ALK.

13. A method for disrupting a condensate in a cell, wherein the condensate comprises a concentrated population of a biomolecule, the method comprising contacting the condensate with a fusion protein comprising a J domain protein and a targeting molecule, wherein the targeting molecule binds the biomolecule.

14. The method of claim 13, wherein when the targeting molecule binds the biomolecule, the J domain protein recruits a chaperone protein to disassemble the condensate.

15. The method of claim 13, wherein the biomolecule comprises a target protein.

16. The method of claim 15, wherein the target protein comprises a truncated mutant, one or more intrinsically disordered regions, or any combination thereof.

17. The method of claim 15, wherein the target protein comprises MLL-AF9, another MLL-fusion condensate, ASXL1, EML4-ALK, or any combination thereof.

18. The method of claim 13, wherein the chaperone protein comprises HSP70.

19. The method of claim 13, wherein the targeting molecule comprises a nanobody or a target protein binding domain.

20. The method of claim 13, wherein the cell is a mammalian cell.


SEQUENCE LISTING (5 sequences)

SEQ ID NO: 1 - protein, 203 aa, synthetic construct
MGKDYYQTLGLARGASDEEIKRAYRRQALRYHPDKNKEPGAEEKFKEIAEAYDVLSDPRKREIFDRYGGGSGGSGGSGGVDEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSS

SEQ ID NO: 2 - protein, 701 aa, synthetic construct
MGKDYYQTLGLARGASDEEIKRAYRRQALRYHPDKNKEPGAEEKFKEIAEAYDVLSDPRKREIFDRYGGGSGGSGGSGGVDAMGLKAAQKTLFPLRSIDDVVRLFAAELGREEPDLVLLSLVLGFVEHFLAVNRVIPTNVPELTFQPSPAPDPPGGLTYFPVADLSIIAALYARFTAQIRGAVDLSLYPREGGVSSRELVKKVSDVIWNSLSRSYFKDRAHIQSLFSFITGTKLDSSGVAFAVVGACQALGLRDVHLALSEDHAWVVFGPNGEQTAEVTWHGKGNEDRRGQTVNAGVAERSWLYLKGSYMRCDRKMEVAFMVCAINPSIDLHTDSLELLQLQQKLLWLLYDLGHLERYPMALGNLADLEELEPTPGRPDPLTLYHKGIASAKTYYRDEHIYPYMYLAGYHCRNRNVREALQAWADTATVIQDYNYCREDEEIYKEFFEVANDVIPNLLKEAASLLEAGEERPGEQSQGTQSQGSALQDPECFAHLLRFYDGICKWEEGSPTPVLHVGWATFLVQSLGRFEGQVRQKVRIVSREAEAAEAEEPWGEEAREGRRRGPRRESKPEEPPPPKKPALDKGLGTGQGAVSGPPRKPPGTVAGTARGPEGGSTAQVPAPAASPPPEGPVLTFQSEKMKGMKELLVATKINSSAIKLQLTAQSQVQMKKQKVSTPSDYTLSFLKRQRKGLPAAKRVKLD

SEQ ID NO: 3 - protein, 175 aa, synthetic construct
MGKDYYQTLGLARGASDEEIKRAYRRQALRYHPDKNKEPGAEEKFKEIAEAYDVLSDPRKREIFDRYGGGSGGSGGSGGVDETSMDSRLQRIHAEIKNSLKIDNLDVNRCIEALDELASLQVTMQQAQKHTEMITTLKKIRRFKVSQVIMEKSTMLYNKFKNMFLVPAAKRVKLD

SEQ ID NO: 4 - protein, 307 aa, synthetic construct
MGKDYYQTLGLARGASDEEIKRAYRRQALRYHPDKNKEPGAEEKFKEIAEAYDVLSDPRKREIFDRYGGGSGGSGGSGGTGMEAIAKYDFKATADDELSFKRGDILKVLNEECDQNWYKAELNGKDGFIPKNYIEMKPHPWFFGKIPRAKAEEMLSKQRHDGAFLIRESESAPGDFSLSVKFGNDVQHFKVLRDGAGKYFLWVVKFNSLNELVDYHRSTSVSRNQQIFLRDIEQVPQQPTYVQALFDFDPQEDGELGFRRGDFIHVMDNSDPNWWKGACHGQTGMFPRNYVTPVNRNVLPPLERLTL

SEQ ID NO: 5 - protein, 192 aa, synthetic construct
MGKDYYQTLGLARGASDEEIKRAYRRQALRYHPDKNKEPGAEEKFKEIAEAYDVLSDPRKREIFDRYGGGSGGSGGSGGTGWFFGKIPRAKAEEMLSKQRHDGAFLIRESESAPGDFSLSVKFGNDVQHFKVLRDGAGKYFLWVVKFNSLNELVDYHRSTSVSRNQQIFLRDIESGRGGSGGSLPPLERLTL